(19) United States
(12) Patent Application Publication
(10) Pub. No.: US 2025/0044302 A1
(43) Pub. Date: Feb. 6, 2025

(54) JUP BIOMARKER FOR THE DIAGNOSIS OF DISEASES OR DISORDERS OF THE FEMALE REPRODUCTIVE TRACT

(71) Applicants: SCAILYTE AG, Basel (CH); UNIVERSITÄT BERN, Bern (CH)

(72) Inventors: Michael D. MUELLER, Bern (CH); Lea DUEMPELMANN, Bern (CH); Thomas ANDRIEU, Bern (CH); Peter NESTOROV, Basel (CH); Cinzia DONATO, Basel (CH); Angelo DUO, Basel (CH); Albulena TOSKA, Basel (CH)

(21) Appl. No.: 18/704,678

(22) PCT Filed: Oct. 26, 2022
(86) PCT No.: PCT/EP2022/080024

§ 371 Date: Apr. 25, 2024

(30) Foreign Application Priority Data
Oct. 26, 2021 (EP) 21204845.8

(51) Int. Cl. G01N 33/68; C12Q 1/6883

(52) U.S. Cl. CPC G01N 33/689; C12Q 1/6883; C12Q 2600/158; G01N 2800/364; G01N 2800/50; G01N 2800/52

(57) ABSTRACT

The invention relates to methods for diagnosing, predicting disease development, disease progression and/or disease outcome, predicting susceptibility to treatment and/or classification in the context of a disease or disorder of the female reproductive tract, wherein JUP alone or in combination with further biomarkers is determined. The invention further relates to pharmaceutical products for use inpatients stratified according to the methods of the invention and to compositions comprising reagents for the detection of the biomarker JUP or of the biomarker JUP and further biomarkers for the diagnosis of a disease or disorder of the female reproductive tract.

The invention relates to methods for diagnosing, predicting disease development, disease progression and/or disease outcome, predicting susceptibility to treatment and/or classification in the context of diseases or disorders of the female reproductive tract, wherein JUP alone or in combination with further biomarkers is determined. The invention further relates to pharmaceutical products for use in patients stratified according to the methods of the invention and to compositions comprising reagents for the detection of the biomarker JUP or of the biomarker JUP and further biomarkers for the diagnosis of diseases or disorders of the female reproductive tract.

Diseases or disorders of the female reproductive tract can occur as a result of disease in one of the reproductive organs. These disorders often present as altered menstruation, pelvic pain, or infertility during the reproductive years. Diseases or disorders of the female reproductive tract include without limitation endometrial cancer, ovarian cancer and endometriosis.

Ovarian cancer is a group of diseases that originates in the ovaries, or in the related areas of the fallopian tubes and the peritoneum. Ovarian cancer is often diagnosed in later stages of the disease (stage III and IV, metastatic) due to its asymptomatic onset. Early detection of ovarian cancer implicates a better response to treatments. Ovarian cancer is relatively rare (0.0146 prevalence in women), however BRCA1 and BRCA2 mutations, and lynch syndrome are high risk factors for ovarian cancer. Ovarian cancer is divided into several subtypes: Invasive epithelial, stromal, germ cell-tumor, fallopian-tumor. 5-years survival rate depends on the subtype and it ranges around 95% for localized tumors, between 50 and 94% in regional tumors and 30-70% in distant settings.

Diagnosis of ovarian cancer comprises several imaging techniques such as MRI and CT-scans as well as blood tests.

Endometriosis is an estrogen dependent disease characterized by the growth of endometrial tissue outside the uterus. These “lesions” can be found throughout the peritoneal cavity, which allows separating largely the disease into three distinct subtypes: superficial peritoneal (SUP), ovarian (OMA or endometrioma), and deep infiltrating endometriosis (DIE) (Chapron, C., et al., Hum Reprod, 2011. 26(8): p. 2028-35). DIE is considered the most severe form of the disease and defined by lesions that infiltrate more than 5 cm into the underlying tissue. Endometriosis is extremely prevalent, occurring in up to 10% of reproductive aged women but also extremely heterogeneous since it can manifest in a variety of symptoms and medical complications. It can lead to significant pelvic pain, subfertility, pregnancy complications (Giudice, L. C., Clinical practice. Endometriosis. N Engl J Med, 2010. 362(25): p. 2389-98) and has been associated with an increased chance of developing ovarian cancer later in life (Wentzensen, N., et al., J Clin Oncol, 2016. 34(24): p. 2888-98).

The pathogenesis of endometriosis is still unclear. The most commonly accepted theory is the one by Sampson of retrograde menstruation originally proposed in 1927 (Sampson, J. A., Am J Pathol, 1927. 3(2): p. 93-110 43). Under this theory, viable endometrial epithelial and stromal cells are refluxed back through the fallopian tubes into the peritoneal cavity during menstruation. These cells are able to avoid immune surveillance and ultimately adhere to and invade the mesothelial cell lining, and proliferate. However, up to 90% of women experience retrograde menstruation but only some of these develop lesions, thus other aberrant biological factors must be involved.

At present, there are no non-invasive diagnostic tests, and direct observation by laparoscopy is the gold standard for diagnosis. If endometriosis is detected during a laparoscopy, these lesions are removed and part of this tissue is examined to confirm the diagnosis. In addition, the peritoneal fluid which would inform about the lesion microenvironment is routinely removed. Influenced by menstrual hormones, both disease progression and the accompanying symptoms are cyclically stimulated. Growth of the lesions can be controlled by hormonal modulation, unfortunately, this represents an inadequate option for women wishing to conceive. Moreover, resistance to hormonal therapy has been recently observed. The prevalence and need for surgery puts an extraordinary strain on health care systems and economic productivity.

Thus, there is a need for improved methods to examine and/or predict disease or disorder of the female reproductive tract-related parameters such as diagnosis, disease development, disease progression, disease outcome and/or susceptibility to treatment.

The above technical problem is solved by the embodiments disclosed herein and as defined in the claims.

Accordingly, the invention relates to, inter alia, the following embodiments:

1. A method for diagnosing a disease or disorder of the female reproductive tract, the method comprising the steps of:
i) determining a protein level of the biomarker JUP in a sample of a female subject; and
ii) diagnosing a disease or disorder of the female reproductive tract based on the determination of i), preferably wherein a protein level above 112 ng/ml serum-equivalent of the biomarker JUP is indicative for a disease or disorder of the female reproductive tract.

2. A method for predicting disease development, disease progression and/or disease outcome of a female subject having a disease or disorder of the female reproductive tract or at risk of having a disease or disorder of the female reproductive tract, the method comprising the steps of:
i) determining a protein level of the biomarker JUP in a sample of a female subject;
ii) comparing the level determined in i) to a prediction reference pattern; and
iii) predicting disease development, disease progression and/or disease outcome of the female subject based on the comparison in step ii), preferably wherein a protein level of the biomarker JUP above the prediction reference pattern is indicative for more likely disease development, more likely disease progression and/or worsening of disease outcome.

3. A method for predicting susceptibility to a treatment for a disease or disorder of the female reproductive tract of a female subject having a disease or disorder of the female reproductive tract or at risk of having a disease or disorder of the female reproductive tract, the method comprising the steps of:
i) determining a protein level of the biomarker JUP in a sample of a female subject;
ii) comparing the level determined in i) to a susceptibility reference pattern; and
iii) predicting susceptibility to a treatment for a disease or disorder of the female reproductive tract of the female subject based on the comparison in step ii), preferably wherein a level of the biomarker JUP above the susceptibility reference pattern is indicative for increased susceptibility to a treatment for a disease or disorder of the female reproductive tract.

4. The method of embodiment 3, wherein the treatment for a disease or disorder of the female reproductive tract is an endometriosis treatment selected from the group of: pain medication, hormonal therapy, fertility treatment and surgery.

5. The method of embodiment 1 to 4, wherein a level of at least the biomarker CA125 is additionally determined in i), preferably wherein the level of the biomarker CA125 is a protein level.

6. The method of embodiment 1 to 5, wherein a level of at least 2, 3, 4, or 5 biomarkers is determined.

7. The method of embodiment 6, wherein determining the level of biomarkers comprises determining at least one biomarker using a nucleic acid detection technique.

8. The method of embodiment 6 or 7 wherein at least one of the biomarkers is selected from the group consisting of: HBA1, HBA2, TRBV2, HBB, TMEM176A, TMEM176B, LTB, KLRC1, RGPD2, IFIT2, JUN and CH25H, preferably wherein the biomarkers comprise at least one biomarker selected from the group consisting of:
a) TRBV2 in CD4+ cytotoxic T cells;
b) TMEM176A and/or TMEM176B in CD14+ monocytes;
c) HBB, HBA1, HBA2, RGPD2 in CD14+ monocytes;
d) CH25H, HBB, LTB, KLRC1 in gamma delta T cells;
e) IFIT2 in CD56+ natural killer cells; and
f) JUN in regulatory T cells.

9. The method of any one of embodiments 1 to 8, wherein sample of a female subject is a sample selected from the group of endometrium sample, menstrual blood sample, vaginal smear sample, a blood sample and/or a cervical smear sample.

10. The method of any one of embodiments 1 to 9, wherein the method is at least partially computer-implemented and wherein the level(s) of the biomarker(s) is/are determined by retrieving data indicative for the level(s) of the biomarker(s).

11. The method of any one of embodiments 1 to 10, wherein the method additionally comprises determining at least one non-molecular marker, preferably wherein the non-molecular marker comprises a marker selected from the group consisting of: age, weight, BMI, gravidity, parity, ethnicity, dysmenorrhea, fertility status, previous laparoscopies, previous use of medication and other gynaecological disorders.

12. The method of embodiment 11, wherein at least 2 biomarkers are determined and wherein the biomarkers comprise or consist of the biomarkers CA125 and JUP, preferably wherein at least 3 biomarkers are determined, wherein the biomarkers comprise or consist of the molecular biomarkers CA125 and JUP and the non-molecular marker dysmenorrhea.

13. The method for predicting of any one of embodiments 2 to 12, wherein the reference pattern is obtained from reference subjects, wherein at least one of the reference subjects is diagnosed with a disease or disorder of the female reproductive tract.

14. The method for predicting of embodiment 13, wherein obtaining the reference pattern from reference subjects comprises a machine-learning technique, preferably a convolutional neural network and/or logistic regression.

15. A method for classification of a female subject having a disease or disorder of the female reproductive tract or at risk of having a disease or disorder of the female reproductive tract into a class, the method comprising the steps of:
a) i) determining a level of the biomarker JUP in a sample of a female subject;
   ii) predicting disease development, disease progression and/or disease outcome of a female subject according to the method of embodiment 2, 5 to 14; and/or
   iii) predicting susceptibility to a treatment for a disease or disorder of the female reproductive tract of a female subject according to the method of any one of embodiments 3 to 14; and
b) classifying the female subject according to the level determined in i), prediction of ii), and/or prediction in iii).

16. The method of embodiment 15, wherein at least one class is indicative of the stage, type and/or severity of the endometriosis.

17. Use of a composition comprising reagents for the detection of the biomarker JUP for the diagnosis of a disease or disorder of the female reproductive tract,

18. Use of the composition of embodiment 17, wherein the disease or disorder of the female reproductive tract is endometriosis.

19. Use of the composition of embodiment 17 or 18, wherein the composition further comprises reagents for the detection of CA125.

20. A pharmaceutical product comprising a compound against a disease or disorder of the female reproductive tract for use in treatment of a female subject that is predicted as susceptible to a treatment for a disease or disorder of the female reproductive tract according to the method for predicting susceptibility to a treatment of embodiment 3 to 16.

21. The pharmaceutical product of embodiment 20, wherein the compound against a disease or disorder of the female reproductive tract is selected from the group of: ibuprofen, naproxen, oxycodone, desogestrel, dienogest, levonorgestrel, clomiphene citrate, gonadotropins, metformin, letrozole and bromocriptine.

22. The method of any one of the embodiments 1 to 16, the use of the composition of any one of the embodiments 17 to 19 or the pharmaceutical product of embodiment 20 or 21, wherein the disease or disorder of the female reproductive tract is endometriosis or ovarian cancer.

23. The method of embodiment 22, the composition of embodiment 22 or the pharmaceutical product of embodiment 22, wherein the disease or disorder of the female reproductive tract is endometriosis.

24. The method of embodiment 23, the composition of embodiment 23 or the pharmaceutical product of embodiment 23, wherein the endometriosis is selected from the group of: peritoneal endometriosis (SUP), endometrioma(s), deeply infiltrating endometriosis (DIE) and abdominal wall endometriosis.

25. The method of embodiment 23 or 24, the composition of embodiment 23 or 24 or the pharmaceutical product of embodiment 23 or 24, wherein the endometriosis is at rASRM II, III, or IV, preferably wherein the endometriosis is at rASRM IV stage.

26. A computer program product comprising instructions to execute the method of any one of embodiments 13 to 16 or 22 to 25, wherein the method is a computer-implemented method and wherein the level of the biomarker JUP in a sample of a female subject is retrieved instead of determined.

27. A kit comprising reagents for the detection of JUP protein and for the detection of at least one RNA marker selected from the group consisting of: TRBV2, HBB, HBA1, HBA2, TMEM176A, TMEM176B, LTB, KLRC1, RGPD2, IFIT2, JUN and CH25H, preferably wherein the RNA biomarker(s) comprise or consist of at least one biomarker selected from the group consisting of:
a) TRBV2 and CD4;
b) i) TMEM176A and/or TMEM176B; and ii) CD14;
c) i) HBB, HBA1, HBA2 and/or RGPD2; and ii) CD14;
d) i) CH25H, HBB, LTB and/or KLRC1 and ii) a gamma delta marker;
e) IFIT2 and CD56; and
f) JUN and a regulatory T cell marker.

28. The kit of embodiment 27 further comprising an instruction manual how to employ the method of any one of the embodiments 7-16, or 22-25.

Accordingly, in one embodiment, the invention relates to a method for diagnosing a disease or disorder of the female reproductive tract, the method comprising the steps of: i) determining a level of the biomarker JUP in a sample of a female subject; and ii) diagnosing a disease or disorder of the female reproductive tract based on the determination of i).

The term “disease or disorder of the female reproductive tract”, as used herein, refers to any disease or disorder of the ovaries, the fallopian tubes, the uterus, the cervix, and/or the vagina and/or any disease or disorder that originates therefrom. In some embodiments, the disease or disorder of the female reproductive tract described herein is a non-transmissible disease or disorder of the female reproductive tract. In some embodiments, the disease or disorder of the female reproductive tract described herein is a non-transmissible disease or disorder of the female reproductive tract, wherein the symptoms include pelvic pain and/or subfertility. In some embodiments, the disease or disorder of the female reproductive tract described herein at least one disease or disorder selected from the group consisting of endometriosis, ovarian cancer and adenomyosis. In some embodiments, the disease or disorder of the female reproductive tract described herein at least one disease or disorder selected from the group consisting of endometriosis, ovarian cancer, adenomyosis and endometrial cancer.

The term “biomarker”, as used herein, refers to a molecule that is part of and/or generated by a cell and serves as an indicator for a disease. Often a biomarker is a gene variant or a gene product, for example an RNA or a polypeptide.

The phrase “determining a level of a biomarker”, as used herein, refers to using a nucleic acid detection technique, a peptide or protein detection technique and/or retrieval of information indicative of a level of a biomarker from a data source.

The term “JUP”, as used herein, refers to a marker encoded by the JUP gene. The marker can be a nucleic acid or a peptide. In some embodiments the biomarker JUP described herein is the protein encoded by the JUP gene also known as plakoglobin, junction plakoglobin or gamma-catenin. The JUP encoded protein is a member of the catenin protein family and homologous to P-catenin.

The term “subject”, as used herein, refers to a mammal, such as a mouse, guinea pig, rat, dog or human. It is understood that the preferred subject is a human.

The term “female subject”, as used herein, refers to a subject having an uterus. In some embodiments the female subject described herein is a pre-menopausal female subject. In some embodiments the female subject described herein is above 18 years old. In some embodiments, the female subject described herein is a subject with unclear or unknown menstrual cycle status.

The term “sample”, as used herein, refers to any sample, where the skilled person is aware that it may comprise a biomarker. In some embodiments, the sample described herein is a tissue sample, a lavage sample or a body fluid sample. In some embodiments, the sample described herein is a FAGS sorted tissue sample.

The present invention relates to a method for the detection of a disease or disorder of the female reproductive tract with high specificity and sensitivity. Other approaches for the detection of a disease or disorder of the female reproductive tract e.g. laparoscopy or by other biomarkers, are invasive, suffer a reduced sensitivity, are not scalable and/or not suitable for early detection. The method of the present invention, on the other hand, allows measuring the level of the biomarker JUP in the blood, other body fluids or tissue.

Furthermore, the inventors found that JUP can identify subjects as having endometriosis, that are not detectable by markers known in the art. As such, new subject populations can be diagnosed and identified, which were not identifiable by the markers known in the art. Certain markers known in the art are dependent on the status of the menstrual cycle. The inventors found that JUP is less dependent on the status of the menstrual cycle, which allows JUP to be used more versatile and in patients, wherein the status of the menstrual cycle has not yet been determined or is not determinable, e.g., because of cycle irregularities.

Accordingly, the invention is at least in part based on the finding that the biomarker JUP is useful for the diagnosis of a disease or disorder of the female reproductive tract.

In certain embodiments, the invention relates to a method for diagnosing a disease or disorder of the female reproductive tract, the method comprising the steps of: i) determining a protein level of the biomarker JUP in a sample of a female subject; and ii) diagnosing a disease or disorder of the female reproductive tract based on the determination of i), preferably wherein a protein level above 112 ng/ml serum-equivalent of the biomarker JUP is indicative for a disease or disorder of the female reproductive tract.

In certain embodiments the invention relates to a method for diagnosing a disease or disorder of the female reproductive tract, the method comprising the steps of: i) determining a level (preferably a protein level) of the biomarker JUP in a sample of a female subject; and ii) diagnosing a disease or disorder of the female reproductive tract based on the determination of i), wherein a level above about 100 ng/ml, about 110 ng/ml, about 112 ng/ml, about 125 ng/ml, about 150 ng/ml, about 175 ng/ml, about 180 ng/ml, about 200 ng/ml, about 225 ng/ml, about 250 ng/ml, about 275 ng/ml, about 300 ng/ml, about 325 ng/ml, about 350 ng/ml, about 375 ng/ml, about 400 ng/ml, serum-equivalent of the biomarker JUP is indicative for a disease or disorder of the female reproductive tract.

The term “serum-equivalent”, as used herein, refers to an equivalent of the JUP encoded protein value that can be obtained from serum (Example 3). The skilled person is aware that the specific threshold depends on the tissue, sample processing, detection method and/or detection antibody.

In certain embodiments the invention relates to a method for predicting disease development, disease progression and/or disease outcome of a female subject having a disease or disorder of the female reproductive tract or at risk of having a disease or disorder of the female reproductive tract, the method comprising the steps of: i) determining a level of the biomarker JUP in a sample of a female subject; ii) comparing the level determined in i) to a prediction reference pattern; and iii) predicting disease development, disease progression and/or disease outcome of the female subject based on the comparison in step ii).

The phrase “risk of having a disease or disorder of the female reproductive tract”, as used herein, refers to having a risk factor for a disease or disorder of the female reproductive tract and/or at least one symptom of a disease or disorder of the female reproductive tract.

The term “reference pattern”, as used herein, refers to a predetermined pattern or a predetermined data point that can be used for comparison and is preferably obtained from reference subjects. The reference pattern comprises at least one data point, such as a data point that can be used as a threshold. In some embodiments, the reference pattern is a machine learning model.

Accordingly, the invention is at least in part based on the finding that the biomarker JUP can be used for predicting disease development, disease progression and/or disease outcome of a disease or disorder of the female reproductive tract.

In certain embodiments the invention relates to a method for diagnosing a female subject with a disease or disorder of the female reproductive tract, the method comprising the steps of: i) determining a level of the biomarker JUP in a sample of a female subject; ii) comparing the level determined in i) to a diagnosis reference pattern; and iii) diagnosing a female subject with a disease or disorder of the female reproductive tract based on the comparison in step ii).

In certain embodiments the invention relates to a method for predicting disease development, disease progression and/or disease outcome of a female subject having a disease or disorder of the female reproductive tract or at risk of having a disease or disorder of the female reproductive tract, the method comprising the steps of: i) determining a level (preferably a protein level) of the biomarker JUP in a sample of a female subject; ii) comparing the level determined in i) to a prediction reference pattern; and iii) predicting disease development, disease progression and/or disease outcome of the female subject based on the comparison in step ii), wherein a level (preferably a protein level) of the biomarker JUP above the prediction reference pattern is indicative for more likely disease development, more likely disease progression and/or worsening of disease outcome.

In certain embodiments the invention relates to a method for monitoring a female subject for a disease or disorder of the female reproductive tract, the method comprising the steps of: i) determining a level of the biomarker JUP in a sample of a female subject at a first time point; ii) determining a level of the biomarker JUP in a sample of a female subject at a second time point; iii) comparing the level determined in i) with the level determined in ii); and iv) monitoring a female subject based on the comparison in step iii), preferably wherein an increase of JUP at the time point ii) compared to the time point i) is indicative for disease presence, disease development, disease progression and/or worsening of predicted disease outcome.

In certain embodiments the invention relates to a method for monitoring a female subject for a disease or disorder of the female reproductive tract, the method comprising the steps of: i) determining a level of the biomarker JUP in a sample of a female subject at a first time point; ii) determining a level of the biomarker JUP in a sample of a female subject at a second time point, iii) comparing the level determined in i) combined with the level determined in ii) to a monitoring reference pattern; and iv) monitoring a female subject based on the comparison in step iii), preferably wherein an increase of JUP above the monitoring reference pattern is indicative for disease presence, disease development, disease progression and/or worsening of predicted disease outcome.

In some embodiments, the invention relates to the method for monitoring described herein or the method(s) for diagnosis described herein, wherein the method is used as a screening method in healthy female subjects to detect disease development.

In certain embodiments the invention relates to a method for predicting susceptibility to a treatment for a disease or disorder of the female reproductive tract of a female subject having a disease or disorder of the female reproductive tract or at risk of having a disease or disorder of the female reproductive tract, the method comprising the steps of: i) determining a level of the biomarker JUP in a sample of a female subject; ii) comparing the level determined in i) to a susceptibility reference pattern; and iii) predicting susceptibility to a treatment for a disease or disorder of the female reproductive tract of the female subject based on the comparison in step ii).

In certain embodiments the invention relates to a method for predicting susceptibility to a treatment for a disease or disorder of the female reproductive tract of a female subject having a disease or disorder of the female reproductive tract or at risk of having a disease or disorder of the female reproductive tract, the method comprising the steps of: i) determining a level of the biomarker JUP in a sample of a female subject; ii) comparing the level determined in i) to a susceptibility reference pattern; and iii) predicting susceptibility to a treatment for a disease or disorder of the female reproductive tract of the female subject based on the comparison in step ii), wherein a level of the biomarker JUP above the susceptibility reference pattern is indicative for increased susceptibility to a treatment for a disease or disorder of the female reproductive tract.

The inventors found that the biomarker JUP or JUP and further biomarkers can be used to determine susceptibility to a treatment before a treatment. In some embodiments, the prediction of susceptibility to a treatment is the prediction of treatment resistance such as treatment resistance to hormonal therapy. Therefore, the method(s) described herein can support treatment decisions.

In certain embodiments the invention relates to the method of the invention, wherein the treatment for a disease or disorder of the female reproductive tract is an anti-cancer treatment. In some embodiments, the anti-cancer treatment described herein is at least one compound selected from the group of carboplatin, avastin, paclitaxel, doxil, methotrexate, lynparza, adriamycin, gemzar, olaparib, doxorubicin, alkeran, paraplatin, zejula, bevacizumab, cisplatin, doxorubicin, gemcitabine, rubraca, cosmegen, hycamtin, topotecan, cyclophosphamide, melphalan, toposar and etopophos.

In certain embodiments the invention relates to the method of the invention, wherein the treatment for a disease or disorder of the female reproductive tract is an endometriosis treatment selected from the group of: hormonal treatment, physiotherapy, surgery, multimodal pain therapy (drugs (e.g. Targin, Oxynorm), TENS machine), individual nutritional counselling (e.g. eating less meat) and complementary medicine.

In certain embodiments the invention relates to the method of the invention, wherein the treatment for a disease or disorder of the female reproductive tract is an endometriosis treatment selected from the group of: pain medication, hormonal therapy, fertility treatment, and surgery.

The term “pain medication”, as used herein, refers to any pain medication that is used to treat symptoms of endometriosis (see e.g. Ruhland, B., et al., 2011, Minerva Ginecol 63: 1-2). In some embodiments, the pain medication described herein is a pain medication selected from the group of ibuprofen, naproxen and oxycodone.

The term “hormonal therapy”, as used herein, refers to any hormonal therapy that is used to treat symptoms of endometriosis (see e.g. Ruhland, B., et al., 2011, Minerva Ginecol 63: 1-2). In some embodiments, the hormonal therapy described herein is progesterone treatment, preferably a gestagen selected from the group of Desogestrel, Dienogest, Levonorgestrel, norethindrone. The hormonal therapy described herein may be administered orally, by implantation, by injection, transdermal or using an intrauterine device.

The term “fertility treatment”, as used herein, refers to any fertility treatment that is used to treat infertility or subfertility in the context of endometriosis (see e.g. Becker, C. M., Gattrell, W. T., Gude, K., & Singh, S. S., 2017, Fertility and sterility, 108(1), 125-136). In some embodiments, the fertility treatment described herein is in vitro fertilization. In some embodiments, the fertility treatment described herein is a treatment selected from the group of: clomiphene citrate, gonadotropins, metformin, letrozole, bromocriptine, follitropin alpha, Intrauterine insemination (IUI) and in vitro fertilization (IVF).

The term “surgery”, as used herein, refers to any surgical procedure that is used to treat symptoms of endometriosis (see e.g. Leonardi, M., et al. 2020, Journal of minimally invasive gynecology, 27(2), 390-407.). In some embodiments, the surgery described herein is a form of surgery selected from the group of conservative surgery, complex surgery, radical surgery, laparotomy and Laparoscopy.

The choice of treatment is particularly relevant in endometriosis, because it can have an effect on disease progression and/or fertility that can be irreversible.

Accordingly, the invention is at least in part based on the finding that the methods described herein enable accurate prediction of susceptibility to a treatment.

Within the present invention, the level of any number of biomarkers may be determined. It is assumed that the sensitivity and specificity increases with the number of biomarkers that are used in the method of the invention. At the same time, the number of biomarkers that can be used in the method of the invention may be limited by the experimental method to determine the levels of the biomarkers and the availability of suitable binding agents.

In certain embodiments the invention relates to the method of the invention, wherein at least one further, at least two, at least three or all of the biomarker(s) selected from Table 1 is/are determined in i).

The inventors found that the determination of JUP in combination with biomarkers from Table 1 can increase sensitivity and/or specificity of the method described herein.

In certain embodiments the invention relates to the methods of the invention, wherein at least the biomarkers JUP and CA125 are determined.

The term “CA125”, as used herein, refers to a marker encoded by the MUC16 gene. The marker can be a nucleic acid or a peptide. In some embodiments the biomarker CA125 described herein is the protein encoded by the MUC16 gene also known as Cancer Antigen 125.

The inventors found that the combination of the biomarkers JUP and CA125 are particularly useful in the context of diseases or disorders of the female reproductive tract. For example while JUP alone is particularly useful as a diagnostic marker of endometriosis in early and peritoneal endometriosis, the addition of CA125 detection improves the sensitivity for more severe forms of endometriosis (DIE and rASRM IV) and extends its utility into other subtypes (endometrioma). Furthermore, JUP and CA125 detect different subject populations and combining these markers results in a synergistic improvement of the predictive value.

Accordingly, the invention is at least in part based on the synergistic effect of JUP and CA125.

In certain embodiments the invention relates to the method of the invention, wherein at least the biomarkers JUP and HE4 are determined.

The term “HE4”, as used herein, refers to a marker encoded by the WFDC2 gene. The marker can be a nucleic acid or a peptide. In some embodiments the biomarker HE4 described herein is the protein encoded by the WFDC2 gene also known as Human Epididymis Protein 4.

The inventors found that the combination of the biomarkers JUP and HE4 are particularly useful in the context of diseases or disorders of the female reproductive tract. For example the biomarker JUP can be indicative for endometriosis and HE4 is indicative for ovarian cancer. The combination of both markers synergistically improves their accuracy and/or sensitivity for diagnosing diseases or disorders of the female reproductive tract and/or predicting parameters thereof.

In certain embodiments the invention relates to the method of the invention, wherein at least the biomarkers JUP and IL-6 are determined.

The term “IL-6”, as used herein, refers to a marker encoded by the IL6 gene. The marker can be a nucleic acid or a peptide. In some embodiments the biomarker IL-6 described herein is the protein encoded by the IL6 gene also known as Interleukin-6.

In certain embodiments the invention relates to the method of the invention, wherein at least the biomarkers JUP and TNF-a are determined.

The term “TNF-a”, as used herein, refers to a marker encoded by the TNF gene. The marker can be a nucleic acid or a peptide. In some embodiments the biomarker TNF-a described herein is the protein encoded by the TNF gene also known as Tumor Necrosis Factor-Alpha.

In certain embodiments the invention relates to the method of the invention, wherein at least 3 biomarkers are determined.

In certain embodiments the invention relates to the method of the invention, wherein at least the biomarkers JUP; CA125 and HE4 are determined.

In certain embodiments the invention relates to the method of the invention, wherein at least the biomarkers JUP; CA125 and IL-6 are determined.

In certain embodiments the invention relates to the method of the invention, wherein at least the biomarkers JUP; CA125 and TNF-a are determined.

In certain embodiments the invention relates to the method of the invention, wherein at least the biomarkers JUP; HE4 and IL-6 are determined.

In certain embodiments the invention relates to the method of the invention, wherein at least the biomarkers JUP; HE4 and TNF-a are determined.

In certain embodiments the invention relates to the method of the invention, wherein at least the biomarkers JUP; TNF-a and IL-6 are determined.

In certain embodiments the invention relates to the method of the invention, wherein at least 4 biomarkers are determined.

In certain embodiments the invention relates to the method of the invention, wherein at least the biomarkers JUP; CA125; HE4 and IL-6 are determined.

In certain embodiments the invention relates to the method of the invention, wherein at least the biomarkers JUP; CA125; HE4 and TNF-a are determined.

In certain embodiments the invention relates to the method of the invention, wherein at least the biomarkers JUP; CA125; IL-6 and TNF-a are determined.

In certain embodiments the invention relates to the method of the invention, wherein at least the biomarkers JUP; HE4; IL-6 and TNF-a are determined.

In certain embodiments the invention relates to the method of the invention, wherein at least 5 biomarkers are determined.

In certain embodiments the invention relates to the method of the invention, wherein at least the biomarkers JUP; HE4; IL-6; TNF-a; CA125 are determined.

In certain embodiments the invention relates to the method of the invention, wherein additionally the level of TREM-1, preferably sTREM-1 is determined in i).

The term “TREM-1”, as used herein, refers to a marker encoded by the TREM1 gene. The marker can be a nucleic acid or a peptide. In some embodiments, the biomarker TREM-1 described herein is the protein encoded by the TREM1 gene also known as transmembrane receptor protein, which is mainly expressed on myeloid cells such as monocytes/macrophages and granulocytes. Upon activation, it triggers and amplifies inflammation through several pathways (Tammaro, Alessandra, et al., 2017, Pharmacology & therapeutics 177: 81-95). Beside the transmembrane protein, a soluble form of TREM-1 (sTREM-1) protein exists, which was found to be increased in patients with inflammatory disease (rheumatoid arthritis), and therefore might be used as biomarker (Inanc, Nevsun, et al., 2021, Scientific reports 11.1: 1-10).

The inventors found that the determination of JUP in combination with further biomarkers can increase sensitivity and/or specificity of the method described herein.

The set of biomarkers described herein may be adapted to obtain adapted panels for use in the method of the invention and to maintain high sensitivity and specificity, wherein the adapted panel consists of the same or a lower number of biomarkers by a method comprising the steps of:
(i) adding one or more biomarkers to a set of biomarkers described herein to obtain an alternative panel;
(ii) placing weight (e.g. as learned by CellCnn) to the biomarkers of the alternative panel by testing the alternative panel on a set of samples with known classification for a disease or disorder of the female reproductive tract associated parameter.
(iii) excluding one or more biomarkers of an absolute weight below the average weight of the biomarkers of the alternative panel to obtain a provisional adapted panel,
(iv) verifying the specificity and selectivity using a validation data set to identify adapted panels.

In step (i) of the method to obtain an adapted panel, the biomarker(s) to be added to the panel can be any biomarker but is/are preferably (a) biomarker(s) selected from the group listed in Table 1. In some embodiments of the invention, (one of) the biomarker(s) to be added to the panel of the current invention is known to be characteristic for a similar biologic function and/or a same cell type as one of the biomarkers of the panel of the current invention. In some embodiments of the invention, the biomarker(s) to be added to the panel may be chosen based on various reasons, including but not limited to economic reasons, availability of reagents and compatibility with the measurement equipment.

In step (ii) of the method to obtain an adapted panel, placing a weight may be done using CellCnn as described in the examples, or using any suitable weighting method known to the skilled person. The full alternative panel and/or a certain number of the biomarkers of the alternative panel can be tested to obtain information for placing a weight to the biomarkers. For example, alternative panel-minus-one controls may be used to obtain information regarding weighting (e.g., as described by Tung, James W et al. Clinics in laboratory medicine vol. 27,3 (2007): 453-68).

In some embodiments of the invention, in step (iii) of the method to obtain an adapted panel, the biomarker with the lowest weight is excluded.

In step (iv) of the method to obtain an adapted panel, the specificity and selectivity of the provisional adapted panel may be verified as described in the examples. Provisional adapted panels that have a specificity and selectivity below a certain specificity and selectivity threshold, are excluded.

In certain embodiments, the invention relates to the method of the invention wherein at least one of the biomarkers is selected from the group consisting of: TRBV2, HBB, HBA1, HBA2, TMEM176A, TMEM176B, LTB, KLRC1, RGPD2, IFIT2, JUN and CH25H.

In certain embodiments, the invention relates to the method of the invention wherein the biomarkers comprise at least one biomarker selected from the group consisting of: a) TRBV2 in CD4+ cytotoxic T cells; b) TMEM176A and/or TMEM176B in CD14+ monocytes; c) HBB, HBA1, HBA2, RGPD2 in CD14+ monocytes; d) CH25H, HBB, LTB, KLRC1 in gamma delta T cells; e) IFIT2 in CD56+ natural killer cells; and f) JUN in regulatory T cells.

The skilled person is aware of how to detect, select and/or separate cytotoxic T cells, monocytes, gamma delta T cells, natural killer cells and regulatory T cells. Any detection, selection and/or separation technique known in the art may be used. In some embodiments the biomarkers CD25 (CD25hi) and FoxP3 are used to detect the regulatory T cells.

The inventors found that the markers described herein could be used to stratify patient populations and to identify subpopulations with distinct disease progression, treatment susceptibility and/or disease outcome.

In certain embodiments the invention relates to the method of the invention, wherein the level(s) of the biomarker(s) comprise(s) an expression level and determining the level in i) comprises a nucleic acid detection technique.

Nucleic acid detection techniques are well known in the art (see e.g. Kolpashchikov, D. M., & Gerasimova, Y. V. (Eds.)., 2013. Nucleic Acid Detection: Methods and Protocols. Humana Press.) In some embodiments, the nucleic acid detection technique described herein is at least on method selected from the group of: qPCR, isothermal amplification techniques, assays with visual or electric signals for point-of-care diagnostics, fluorescent in situ hybridization and signal amplification techniques.

Therefore, JUP and/or other biomarkers described herein may be detected on the DNA or RNA level, preferably mRNA level.

In certain embodiments the invention relates to the method of the invention, wherein the level(s) of the biomarker(s) comprise(s) a protein level.

In some embodiments, JUP is detected on protein level, while other biomarkers are detected by a nucleic acid detection technique. In other embodiments, several or all biomarkers are detected on protein level.

The protein level can be determined by any method known in the art. In some embodiments, the protein level described herein is determined by an antibody-based assay. That is, any assay that comprises the use of antibodies and is suitable for determining the expression level of a biomarker may be used in the present invention. Preferably, antibodies are used that bind directly to the biomarker. Within the present invention, the antibodies are preferably labeled to facilitate detection and/or quantification of a biomarker. For example, antibodies may be labeled with a fluorophore to allow detection and/or quantification of biomarkers in flow cytometry-based assays or metal isotopes to allow detection and/or quantification of biomarkers in mass cytometry-based assays. In some embodiments, the invention relates to the method according to the invention, wherein the antibody-based assay is an antibody-based flow cytometry or mass cytometry assay. In some embodiments, the protein level described herein is determined by ELISA, preferably multiplexed ELISA.

In certain embodiments the invention relates to the method of the invention, wherein sample of a female subject is a sample selected from the group of endometrium sample, menstrual blood sample, vaginal smear sample, a blood sample and/or a cervical smear sample.

The inventors found that method of the invention is particularly sensitive, specific and or minimally invasive when using certain types of samples.

In certain embodiments the invention relates to the method of the invention, wherein the method is at least partially computer-implemented and wherein the level(s) of the biomarker(s) is/are determined by retrieving data indicative for the level(s) of the biomarker(s).

The inventors found that the method of the invention can be used on databases and/or data of samples. This enables scalability and/or separating the sample obtainment procedure from the interpretation of the sample.

In certain embodiments the invention relates to the method of the invention, wherein the method additionally comprises determining at least one non-molecular marker.

The term “non-molecular marker”, as used herein, refers to any marker that describes a characteristic of a subject that is not a nucleic acid, peptide or protein.

In certain embodiments the invention relates to the method of the invention, wherein the method additionally comprises at determining least one non-molecular marker, wherein the non-molecular marker comprises a marker selected from the group consisting of: age, weight, BMI, gravidity, parity, ethnicity, dysmenorrhea, fertility status, previous laparoscopies, previous use of medication and other gynaecological disorders.

The term “other gynaecological disorder”, as used herein, refers to any gynaecological disorder other than the disease or disorder of the female reproductive tract that is diagnosed, predicted and/or classified according to the method of the invention. In some embodiments, the other gynaecological disorder described herein is a gynaecological disorder other than endometriosis and ovarian cancer. In some embodiments, the other gynaecological disorder described herein is a gynaecological disorder other than endometriosis. In some embodiments, the other gynaecological disorder described herein is a gynaecological disorder other than ovarian cancer.

The inventors found that non-molecular markers can improve the sensitivity and/or specificity of the method of the invention.

In certain embodiments the invention relates to the method of the invention, wherein the reference pattern is obtained from reference subjects, wherein at least one of the reference subjects is diagnosed with a disease or disorder of the female reproductive tract.

The term “the reference pattern” can context-dependent refer to the prediction reference pattern or the susceptibility reference pattern.

The inventors found that using data of subjects suffering from a disease or disorder of the female reproductive tract can be used as a reference.

Accordingly, the invention is at least in part based on the finding that data from diseased subjects is particularly useful for the reference pattern in the methods described herein.

In certain embodiments, the invention relates to the method of the invention, wherein at least 3 biomarkers are determined and wherein the biomarkers comprise or consist of the molecular biomarkers CA125 and JUP and the non-molecular marker dysmenorrhea.

The inventors found that JUP and CA125 determine different patient populations and that in combination with dysmenorrhea, the predictive performance can be surprisingly improved.

Accordingly, the invention is at least in part based on the finding that the combination of these three markers results in an improved prediction of a disease or disorder of the female reproductive tract such as endometriosis.

In certain embodiments the invention relates to the method of the invention, wherein the reference pattern is obtained from reference subjects, wherein at least one of the reference subjects is diagnosed with a disease or disorder of the female reproductive tract and at least one of the reference subjects is not having a disease or disorder of the female reproductive tract.

In certain embodiments the invention relates to the method of the invention, wherein obtaining the reference pattern from reference subjects comprises a machine-learning technique.

The term “machine-learning technique”, as used herein, refers to a computer-implemented technique that enables automatic learning and/or improvement from an experience (e.g., training data and/or obtained data) without the necessity of explicit programming of the lesson learned and/or improved. In some embodiments, the machine learning technique comprises at least one technique selected from the group of Logistic regression, CART, Bagging, Random Forest, Gradient Boosting, Linear Discriminant Analysis, Gaussian Process Classifier, Gaussian NB, Linear, Lasso, Ridge, ElasticNet, partial least squares, KNN, DecisionTree, SVR, AdaBoost, GradientBoost, neural net and ExtraTrees.

The inventors found that machine-learning techniques provide an efficient and/or unbiased way to identify patterns predictive for disease- and treatment-related parameters.

In certain embodiments the invention relates to the method of the invention, wherein obtaining the reference pattern from reference subjects comprises a convolutional neural network and/or logistic regression.

The CellCnn convolutional neural network has been described previously (Arvaniti, E., Claassen, M., 2017, Nat Commun 8, 14825; Bodenmiller et al., Nat Biotechnol, 2012, 30(9), 858-867; Amir et al., Nat Biotechnol, 2013, 31(5), 545-552; Levine et al., Cell, 2015, 162(1), 184-197; Horowitz et al., Sci Transl Med, 2013, 5(208), 208ra145) and is publicly available (https://github.com/eiriniar/CellCnn). Further, it is described in the Examples how the CellCnn convolutional neural network may be used in the context of the invention.

In certain embodiments the invention relates to a method for classification of a female subject having a disease or disorder of the female reproductive tract or at risk of having a disease or disorder of the female reproductive tract into a class, the method comprising the steps of: a) i) determining a level of the biomarker JUP in a sample of a female subject; ii) predicting disease development, disease progression and/or disease outcome of a female subject according to the method of the invention; and/or iii) predicting susceptibility to a treatment for a disease or disorder of the female reproductive tract of a female subject according to the method of the invention; and b) classifying the female subject according to the level determined in i), prediction of ii), and/or prediction in iii).

The inventors found that the method described herein can be used for classifying subject population. Using this classification method, established classes (e.g. stages or grades) are obtainable, are more standardized and/or with reduced effort.

Furthermore, the classification method of the invention may be used to identify novel classes subject populations with distinct properties.

In certain embodiments the invention relates to the method of the invention, wherein at least one class is indicative of the stage of the endometriosis.

In certain embodiments the invention relates to the method of the invention, wherein at least one class is indicative of the severity of the endometriosis.

In certain embodiments the invention relates to the method of the invention, wherein at least one class is indicative of the type of the endometriosis.

In certain embodiments the invention relates to the method of the invention, wherein at least one class is indicative of the stage and severity of the endometriosis.

In certain embodiments the invention relates to the method of the invention, wherein at least one class is indicative of the stage and type of the endometriosis.

In certain embodiments the invention relates to the method of the invention, wherein at least one class is indicative of the type and severity of the endometriosis.

In certain embodiments the invention relates to the method of the invention, wherein at least one class is indicative of the stage, type and severity of the endometriosis.

The term “stage of the endometriosis”, as used herein, refers to an established stage of endometriosis such as the four rASRM stages (Rock, J. A., & ZOLADEX Endometriosis Study Group, 1995, Fertility and sterility, 63(5), 1108-1110).

In certain embodiments the invention relates to a composition comprising reagents for the detection of the biomarker JUP for the diagnosis of a disease or disorder of the female reproductive tract.

In certain embodiments the invention relates to a composition comprising reagents for the detection of the biomarker JUP and at least one, at least two at least three, at least four biomarkers from Table 1 for the diagnosis of a disease or disorder of the female reproductive tract.

In certain embodiments the invention relates to a composition comprising or consisting of the reagents for the detection of the biomarkers a) JUP and CA125; b) JUP and HE4; c) JUP and IL-6; or d) JUP and TNF-a for the diagnosis of a disease or disorder of the female reproductive tract.

In certain embodiments the invention relates to a composition comprising or consisting of the reagents for the detection of the biomarkers a) JUP and CA125; b) JUP and HE4; c) JUP and IL-6; d) JUP and S100A12 or e) JUP and TNF-a for the diagnosis of a disease or disorder of the female reproductive tract.

In certain embodiments the invention relates to a composition comprising or consisting of the reagents for the detection of the biomarkers a) JUP, CA125 and HE4; b) JUP, CA125 and IL-6; c) JUP, CA125 and TNF-a; d) JUP, HE4 and IL-6; e) JUP, HE4 and TNF-a; or f) JUP, TNF-a, IL-6 for the diagnosis of a disease or disorder of the female reproductive tract.

In certain embodiments the invention relates to a composition comprising or consisting of the reagents for the detection of the biomarkers a) JUP, CA125 and HE4; b) JUP, CA125 and IL-6; c) JUP, CA125 and TNF-a; d) JUP, HE4 and IL-6; e) JUP, HE4 and TNF-a; f) JUP, TNF-a and IL-6; g) JUP, S100A12 and CA125; h) JUP, S100A12, HE4; i) JUP, S100A12 and TNF-a; or i) JUP, S100A12 and IL-6 for the diagnosis of a disease or disorder of the female reproductive tract.

In certain embodiments the invention relates to a composition comprising or consisting of the reagents for the detection of the biomarkers a) JUP, CA125, HE4 and IL-6; b) JUP, CA125, HE4 and TNF-a; c) JUP, CA125, IL-6 and TNF-a; or d) JUP, HE4, IL-6 and TNF-a for the diagnosis of a disease or disorder of the female reproductive tract.

In certain embodiments the invention relates to a composition comprising or consisting of the reagents for the detection of the biomarkers a) JUP, CA125, HE4 and IL-6; b) JUP, CA125, HE4 and TNF-a; c) JUP, CA125, IL-6 and TNF-a; d) JUP, HE4, IL-6 and TNF-a; e) JUP, S100A12, HE4 and IL-6; f) JUP, S100A12, HE4 and TNF-a; g) JUP, S100A12, IL-6 and TNF-a; h) JUP, CA125, S100A12 and IL-6; i) JUP, CA125, S100A12 and TNF-a; j) JUP, CA125, HE4 and S100A12; or k) JUP, HE4, IL-6 and S100A12 for the diagnosis of a disease or disorder of the female reproductive tract.

In certain embodiments the invention relates to a composition comprising or consisting of the reagents for the detection of the biomarkers JUP, HE4, IL-6, TNF-a, CA125 for the diagnosis of a disease or disorder of the female reproductive tract.

In certain embodiments the invention relates to a composition comprising or consisting of the reagents for the detection of the biomarkers a) JUP, HE4, IL-6, TNF-a, and CA125; b) JUP, HE4, IL-6, TNF-a, and S100A12; c) JUP, HE4, IL-6, CA125 and S100A12; d) JUP, HE4, TNF-a, CA125 and S100A12; or e) JUP, IL-6, TNF-a, CA125 and S100A12 for the diagnosis of a disease or disorder of the female reproductive tract.

In certain embodiments the invention relates to a composition comprising or consisting of the reagents for the detection of the biomarkers JUP, S100A12, HE4, IL-6, TNF-a, CA125 for the diagnosis of a disease or disorder of the female reproductive tract.

In certain embodiments, the invention relates to the use of a composition described herein for the diagnosis of a disease or disorder of the female reproductive tract.

In certain embodiments, the invention relates to the use of a composition comprising reagents for the detection of the biomarker JUP for the diagnosis of a disease or disorder of the female reproductive tract.

In certain embodiments, the invention relates to use of the composition of the invention, wherein the disease or disorder of the female reproductive tract is endometriosis.

In certain embodiments, the invention relates to use of the composition of the invention, wherein the composition further comprises reagents for the detection of CA125.

In certain embodiments the invention relates to a pharmaceutical product comprising a compound against a disease or disorder of the female reproductive tract for use in treatment of a female subject that is predicted and/or classified as susceptible to a treatment for a disease or disorder of the female reproductive tract according to the method(s) of the invention.

The term “pharmaceutical product”, as used herein, refers to a preparation which is in such form as to permit the biological activity of an active ingredient contained therein to be effective, and which contains no additional components which are unacceptably toxic to a subject to which the formulation would be administered.

The term “compound against a disease or disorder of the female reproductive tract”, as used herein, refers to any compound that is known to be effective in the treatment of disease or disorder of a female reproductive tract and/or symptoms thereof.

The inventors found that, using the method(s) of the invention, subject populations that are particularly sensitive to certain pharmaceutical products can be identified. As such, the pharmaceutical products have a surprisingly enhanced risk/benefit ratio in this/these subject population(s).

In certain embodiments the invention relates to the pharmaceutical product of the invention, wherein the compound against a disease or disorder of the female reproductive tract is an anti-cancer treatment.

In certain embodiments the invention relates to the pharmaceutical product of the invention, wherein the compound against a disease or disorder of the female reproductive tract is selected from the group of: ibuprofen, naproxen, oxycodone, desogestrel, dienogest, levonorgestrel, clomiphene citrate, gonadotropins, metformin, letrozole and bromocriptine.

In certain embodiments the invention relates to a method of treatment, the method comprising the steps of: 1) classifying and/or predicting susceptibility to a treatment for a disease or disorder of the female reproductive tract of a female subject having a disease or disorder of the female reproductive tract or at risk of having a disease or disorder of the female reproductive tract according to the method of the invention; and 2) treating the female subject with a treatment for a disease or disorder of the female reproductive tract, wherein the choice of a treatment for a disease or disorder of the female reproductive tract depends on the predicted susceptibility and/or the classification of susceptibility in step (1).

In certain embodiments the invention relates to a method of treatment, the method comprising the steps of: 1) classifying and/or predicting susceptibility to a treatment for a disease or disorder of the female reproductive tract of a female subject having a disease or disorder of the female reproductive tract or at risk of having a disease or disorder of the female reproductive tract according to the method of the invention; and 2) treating the female subject with at least one disease or disorder of the female reproductive tract treatment selected from the group of anti-cancer treatment, pain medication, hormonal therapy, fertility treatment and surgery, wherein the choice of a disease or disorder of the female reproductive tract treatment depends on the predicted susceptibility and/or the classification of susceptibility in step (1).

In certain embodiments the invention relates to a method of treatment, the method comprising the steps of: 1) classifying and/or predicting susceptibility to a treatment for a disease or disorder of the female reproductive tract of a female subject having a disease or disorder of the female reproductive tract or at risk of having a disease or disorder of the female reproductive tract according to the method of the invention; and 2) treating the female subject with a pharmaceutical product selected from the group of: ibuprofen, naproxen, oxycodone, desogestrel, dienogest, levonorgestrel, clomiphene citrate, gonadotropins, metformin, letrozole and bromocriptine, wherein the choice of pharmaceutical product depends on the predicted susceptibility and/or the classification of susceptibility in step (1).

In certain embodiments the invention relates to the method of the invention, the composition of the invention or the pharmaceutical product of the invention, wherein the disease or disorder of the female reproductive tract is endometriosis, ovarian cancer, adenomyosis and/or endometrial cancer.

The term “ovarian cancer”, as used herein, refers to a condition characterized by anomalous rapid proliferation of ovarian cells and/or of cells in the ovarian area of a subject. In some embodiments, the ovarian cancer described herein is a primary ovarian cancer.

The term “adenomyosis”, as used herein, refers to a condition characterized by cell growth within the uterus characterized by cell growth that causes the uterus to thicken and/or enlarge.

The term “endometrial cancer”, as used herein, refers to a condition characterized by anomalous rapid proliferating cells in the tissue lining the uterus. In some embodiments, the endometrial cancer described herein is a primary endometrial cancer.

In certain embodiments the invention relates to the method of the invention, the composition of the invention or the pharmaceutical product of the invention, wherein the disease or disorder of the female reproductive tract is endometriosis, ovarian cancer, and/or adenomyosis.

The inventors found that the means and methods of the invention are particularly sensitive and/or specific in the context of endometriosis, ovarian cancer and/or in distinguishing between these indications.

In certain embodiments the invention relates to the method of the invention, the composition of the invention or the pharmaceutical product of the invention, wherein the disease or disorder of the female reproductive tract is endometriosis, ovarian cancer, and/or endometrial cancer.

In certain embodiments the invention relates to the method of the invention, the composition of the invention or the pharmaceutical product of the invention, wherein the disease or disorder of the female reproductive tract is endometriosis and/or ovarian cancer.

In certain embodiments the invention relates to the method of the invention, the composition of the invention or the pharmaceutical product of the invention, wherein the disease or disorder of the female reproductive tract is endometriosis.

The term “endometriosis”, as used herein, refers to a disease of the female reproductive system in which cells similar to those in the endometrium, the layer of tissue that normally covers the inside of the uterus, grow outside the uterus.

Risk factors for endometriosis include without limitation genetic risk factors (e.g. a relative diagnosed with endometriosis and/or a mutation in one or more of the WNT4, GREB1/FN1, ID4, 7p15.2, CDKN2BAS, 10q26, VEZT, MUC16 genes/regions), a history of symptoms of endometriosis and environmental toxins (e.g. exposure to estrogen, exposure to dioxin or obstruction of menstrual outflow).

The inventors found that the means and methods of the invention are particularly sensitive and/or specific in the context of endometriosis.

In certain embodiments the invention relates to the method of the invention, the composition of the invention or the pharmaceutical product of the invention, wherein the disease or disorder of the female reproductive tract is endometriosis and wherein CA125 instead of JUP is used.

Previous research suggested that CA125 alone is insufficient as a biomarker for endometriosis (Muyldermans, M., 1995, Human reproduction update, 1(2), 173-187). The inventors found that CA125 alone is useful in the context of endometriosis and further identified combinations comprising CA125 that are surprisingly useful in identifying diagnosis, predicting disease development, predicting disease progression, predicting susceptibility to a treatment and/or identifying (a) subject population(s) that can be treated by the composition(s) described herein.

In certain embodiments the invention relates to the method of the invention, the composition of the invention or the pharmaceutical product of the invention, wherein the endometriosis is selected from the group of: peritoneal endometriosis, endometriomas, deeply infiltrating endometriosis and abdominal wall endometriosis.

The inventors found that the biomarker JUP or the biomarker JUP and further biomarkers are particularly altered in certain types of endometriosis. Accordingly, the invention is at least in part based on the finding that the methods described herein are particularly sensitive and/or specific in certain types of endometriosis.

In certain embodiments the invention relates to the method of the invention, the composition of the invention or the pharmaceutical product of the invention, wherein the endometriosis is at rASRM I stage. In certain embodiments the invention relates to the method of the invention, the composition of the invention or the pharmaceutical product of the invention, wherein the endometriosis is at rASRM II stage. In certain embodiments the invention relates to the method of the invention, the composition of the invention or the pharmaceutical product of the invention, wherein the endometriosis is at rASRM III stage. In certain embodiments the invention relates to the method of the invention, the composition of the invention or the pharmaceutical product of the invention, wherein the endometriosis is at rASRM IV stage. In certain embodiments the invention relates to the method of the invention, the composition of the invention or the pharmaceutical product of the invention, wherein the endometriosis is at rASRM III or IV stage. In certain embodiments the invention relates to the method of the invention, the composition of the invention or the pharmaceutical product of the invention, wherein the endometriosis is at rASRM II or III stage. In certain embodiments the invention relates to the method of the invention, the composition of the invention or the pharmaceutical product of the invention, wherein the endometriosis is at rASRM I or II stage. In certain embodiments the invention relates to the method of the invention, the composition of the invention or the pharmaceutical product of the invention, wherein the endometriosis is at rASRM I, II or III stage.

In certain embodiments the invention relates to the method of the invention, the composition of the invention or the pharmaceutical product of the invention, wherein the endometriosis is at rASRM II, III, or IV stage.

The inventors found that the biomarker JUP or the biomarker JUP and further biomarkers are particularly altered in later stages of endometriosis.

Accordingly, the invention is at least in part based on the finding that the methods described herein are particularly sensitive and/or specific in later stages of endometriosis.

In certain embodiments the invention relates to the method of the invention, the composition of the invention or the pharmaceutical product of the invention, wherein the endometriosis is at rASRM IV stage.

In certain embodiments the invention relates to a computer program product comprising instructions to execute the method of the invention, wherein the method is a computer-implemented method and wherein the level of the biomarker JUP in a sample of a female subject is retrieved instead of determined.

The computer program product described herein may comprise computer-readable program instructions that can be downloaded to respective computing/processing devices from a computer-readable storage medium or to an external computer or external storage device via a network.

Computer-readable program instructions for carrying out operations of the present invention may be assembler instructions, instruction-set-architecture (ISA) instructions, machine instructions, machine-dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages.

The computer-readable program instructions may execute entirely on the user's computer, partly on the user's computer, as a stand-alone software package, partly on the user's computer and partly on a remote computer or entirely on the remote computer or server.

In certain embodiments, the invention relates to a kit comprising reagents for the detection of JUP protein and for the detection of at least one RNA marker selected from the group consisting of: TRBV2, HBB, HBA1, HBA2, TMEM176A, TMEM176B, LTB, KLRC1, RGPD2, IFIT2, JUN and CH25H, preferably wherein the RNA biomarker(s) comprise or consist of at least one biomarker selected from the group consisting of: a) TRBV2 and CD4; b) i) TMEM176A and/or TMEM176B; and ii) CD14; c) i) HBB, HBA1, HBA2 and/or RGPD2; and ii) CD14; d) i) CH25H, HBB, LTB and/or KLRC1 and ii) a gamma delta marker; e) IFIT2 and CD56; and f) JUN and a regulatory T cell marker such as CD25 (CD25hi) and/or FoxP3.

In order to carry out the method of the invention, the kit can be prepared by collecting necessary reagents. In a further embodiment, the invention relates to a kit, in particular a kit for use in detecting JUP protein and optionally further biomarkers. Such a kit may further comprise one or more containers for receiving a sample, protein detection components (e.g. ELISA components), and/or nucleic acid (amplification and) detection components (e.g. digestion enzymes, buffer, primer and/or probes).

In a particularly preferred embodiment of the present invention, the kits (to be prepared in context) of this invention or the methods and uses of the invention may further comprise or be provided with (an) instruction manual(s). For example, said instruction manual(s) may guide the skilled person (how) to employ the kit of the invention in the uses provided herein and in accordance with the present invention. Particularly, said instruction manual(s) may comprise guidance to use or apply the herein provided methods or uses.

“a,” “an,” and “the” are used herein to refer to one or to more than one (i.e., to at least one, or to one or more) of the grammatical object of the article.

“or” should be understood to mean either one, both, or any combination thereof of the alternatives.

“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

The terms “about” or “approximately”, as used herein, refer to “within 20%”, more preferably “within 10%”, and even more preferably “within 5%”, of a given value or range.

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While embodiments of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF FIGURES

FIG. 1 RNA Expression of JUP in PBMCs detected through single-cell RNA-sequencing
A Differential testing of Single-cell RNA-sequencing data on 30 PBMC samples shows that JUP is differentially expressed between Control and Endometriosis
B Violin plot of JUP RNA expression CTL (Control) vs. Endo (endometriosis)
C UMAP plot of CTL (Control) vs. Endo (endometriosis)

FIG. 2 Expression of soluble JUP in sera of non-endometriosis (CTL) or endometriosis (Endo) patients (ELISA). Number of samples is indicated where applicable (n number).
A. Overall expression of soluble JUP in sera of CTL and Endo patients through ELISA.
B. Receiving Operator Characteristics (ROC) curve of JUP showing the accuracy of JUP as biomarker for endometriosis.
C-E. On the left, ROC curves of JUP cutoffs considering all menstrual cycle phases; from top to bottom, low, middle and high cutoff of JUP. On the right, bar plots showing the number of Endo patients (1) identified using the cutoff of reference on the left and compared to CTL (0).
F-H. On the left, ROC curves of JUP cutoffs considering the secretory phase only; from top to bottom, low, middle and high cutoff of JUP. On the right, bar plots showing the number of Endo patients (1) identified using the cutoff of reference on the left and compared to CTL (0).
I. Expression of soluble JUP in sera of CTL and Endo patients at different menstrual cycle phase and obtained through ELISA analysis.
J. Expression of soluble JUP in sera of CTL (0) and Endo (1) patients at different stages of the diseases (rARSM I-IV).

FIG. 3 Expression of soluble CA125, and performance in combination with JUP in sera of non-endometriosis (CTL) or endometriosis (Endo) patients (ELISA). Number of replicates is indicated where applicable (n number).
A. Overall expression of soluble CA125 in sera of CTL and Endo patients through ELISA.
B. Receiving Operator Characteristics (ROC) curve of CA125 high cutoff showing the accuracy of CA125 as biomarker for endometriosis.
C. Correlation between the expression of JUP (>380 ng/ml) and CA125 (>35 U/ml) in endometriosis (Endo) and non-endometriosis (CTL) samples of all menstrual cycle phases.

FIG. 4 Expression of soluble HE4, IL-6, TNFa and CRP in sera of non-endometriosis (CTL) or endometriosis (Endo) patients (ELISA). Number of replicates is indicated where applicable (n number).
A. Expression of soluble JUP and soluble HE4 in sera of CTL and Endo patients showing their correlation.
B. Expression of soluble JUP and soluble TNF-alpha (TNF-a) in sera of CTL and Endo patients showing their correlation.
C. Expression of soluble JUP and soluble IL-6 in sera of CTL and Endo patients showing their correlation.
D. Expression of soluble JUP and soluble CRP in sera of CTL and Endo patients showing their correlation.

FIG. 5 Heatmap of the correlation of JUP with all markers of endometriosis in all menstrual cycle phases.

FIG. 6 Factors influencing serum JUP:
A Endometriosis effect, Two Way Anova p=0.0131, *, Endometriosis (Endo); Adenomyosis (Adeno)
B Menstrual phase effect, Two Way Anova p=0.2458, n.s, Proliferative phase (Prolif); Secretory phase (Secr)
C Age effect in Endometriosis patients (Pearson's r=0.287; p=0.013, *, n=75)
D BMI effect in Endometriosis patients (Pearson's r=0.292; p=0.027, *, n=57)
E Stage effect, One Way Anova p=0.103, n.s. (Adenomyosis excluded)
F Endometrioma effect, One Way Anova p=0.030, * (Adenomyosis excluded); Endometrioma (OMA)
G Stage effect, One Way Anova p=0.0184, *, (Adenomyosis included)
H Endometrioma effect, One Way Anova p=0.007, **, (Adenomyosis included)

FIG. 7 JUP serum level in comparison with inflammatory and competitor endometriosis markers:
A JUP vs. CA125 in Control (CTL) and Endo (endometriosis)
B JUP vs. IL-6 in Control (CTL) and Endo (endometriosis)
C JUP vs. CRP in Control (CTL) and Endo (endometriosis)
D JUP vs. S100A12 in Control (CTL) and Endo (endometriosis)
E JUP vs. TNFalpha in Control (CTL) and Endo (endometriosis)
F JUP vs. HE-4 in Control (CTL) and Endo (endometriosis)

FIG. 8 TMEM176A and TMEM176B in CD14+ monocytes A: violin plot; B: feature plot

FIG. 9 RGPD2 in CD14+ monocytes A: violin plot; B: feature plot

FIG. 10 LTB in gamma delta T cells A: violin plot; B: feature plot

FIG. 11 JUN in T reg cells A: violin plot; B: feature plot

### EXAMPLES

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention.

### Example 1

### Clinical Study Design

Phase I Discovery: An open label study for the discovery of biomarkers for the diagnosis and prognosis of endometriosis.

### Study Population

Patients were recruited at the Frauenklinik, Bern after approval of this application. Inclusion criteria for this study include women who provide Informed Consent and are scheduled for laparoscopic surgery for reasons including suspected endometriosis, tubal ligation, idiopathic infertility or other gynaecological pathologies as part of their planned clinical treatment. Women above 18 years old of all ethnicities and sociodemographic backgrounds were included. Patients with other pre-existing inflammatory diseases, pregnancy, malignancy or undergoing emergency surgery were excluded.

Blood and/or endometrial biopsy were isolated from a total of 99 patients with suspected endometriosis immediately before surgery.

### Clinical Investigation Objectives

### Primary:

The primary objective of this project is to identify a significant biomarker signature in the blood of women with or without endometriosis, which contributes to an early identification of patients suffering from endometriosis.

### Secondary:

The secondary objective of this project is to identify a significant biomarker signature or a significant biological variation to make a prognosis for women with endometriosis.

### Data Types

Clinical Parameters: Full, anonymized clinical data e.g. age, weight, BMI, gravidity and parity, ethnicity, previous laparoscopies, use of (hormonal) medication, other gynaecological disorders, etc.

Single-cell RNA sequencing: RNA expression profile performed on PBMCs from 32 patients.

ELISA panels: Soluble protein expression measurements performed on sera from 67 patients

### Inclusion Criteria

Signed and dated informed consent.

Pre-menopausal women >18 years.

Women undergoing laparoscopic surgery.

Good general health as proven by medical history, physical and gynecological examinations, and laboratory test results.

The study was performed on patients in the menstrual, proliferative or secretory phase of the menstrual cycle. The sera of patients were taken right before the operation to determine the progesterone levels and to confirm the menstrual cycle phase.

The study has no influence on further therapeutic steps.

### Exclusion Criteria

Patient unlikely to cooperate or legally incompetent, including patients who are institutionalized by court or official order. Any condition which could interfere with the patient's ability to comply with the study.

Patients who are pregnant or lactating.

Patients suffering from other inflammatory diseases.

Patients with diagnosed oncologic disease previa laparoscopy.

Patients using hormonal contraceptives or an IUD within 3 months prior to the laparoscopy.

As additional selection criteria, we balanced the samples according to the patient's cycle phase and included diverse endometriosis stages (rASRM I-IV), endometriosis types (DIE, endometrioma, peritoneal, or combination) and pain scores.

### Methodology

The biomarkers were identified through analysis of the patient cohort data. PBMCs from the blood of 32 patients and the sera from the blood of 70 patients were analyzed using single-cell RNA sequencing or ELISA, respectively, to allow a comprehensive characterization of the patient's immune system.

Whole blood: Whole blood was collected, prepared and stored as serum, plasma and PBMC suspension.

Gene expression: Cell capture and cDNA library generation was performed using a Chromium system (10X Genomics). The cDNA library was sequenced using an Illumina platform.

Protein expression: Secreted proteins were quantified by commercial and home-developed enzyme linked immunosorbent assays (ELISA) using standard single or multiplexed procedures.

### Patient Data

Collected medical data was curated into a format for integration into our internal deep learning platform ScaiVision™ or another suitable data analysis workflow that uses patient data as a tool to identify disease-related molecular profiles/or cell identity biomarkers.

### Data Analytics

Data pre-processing
Quality control to check for technical or batch effects
Automated cell-type annotation
Supervised discovery of predictive biomarkers using pseudo bulk gene expression analysis

The goal is to validate at least one soluble biomarker that is predictive of one or more of the stated endpoints with a sensitivity and specificity >=80%

### Project Duration

24 months

### Statistical Analysis

We identified a set of biomarkers that define a signature present specifically in samples of endometriosis patients using a machine learning algorithm ScaiNet based on CellCnn (Arvaniti and Claasen 2017). To create an independent validation set, we randomly split 40% of the data of the 32 PBMC samples before starting network training. We considered any biomarker profile passing the accuracy significance threshold (of >80%) as a potential candidate. 5-fold cross-validation was used to evaluate the reproducibility of the CellCnn algorithm on the specific dataset.

Receiver operating characteristic (ROC) curves were generated to visualize the tradeoff between high sensitivity and high specificity and to determine the threshold laboratory value that separates a clinical diagnosis of “normal” from one of “endometriosis”. The results of the ELISA were analyzed using Wilson/Brown method, confidence interval of 95%, and GraphPad Prism 9.2.0 or MetaboAnalyst 5.0 softwares. The sensitivity and specificity of the ELISAs were estimated from each cutoff value, and cutoff values with the minimum of utility (sensitivity >50%), optimal utility (upper left corner) and high specificity (highest specificity) were selected.

### Explanation of Work Done

Biomarker discovery was carried out on a cohort of PBMC samples collected from patients directly prior to a planned surgery which were subsequently diagnosed with endometriosis or non-endometriosis. Single-cell RNA sequencing was performed on 32 of the isolated PBMC samples measuring detectable levels of RNA transcripts in single cells of the blood.

The workflow consists of steps for quality control of the raw read sequences, transcript quantification, quality control of the samples on a gene- and cell-level, normalisation, dimension reduction and sample class prediction using ScaiNet. The workflow is embedded in the workflow management engine Snakemake (Köster, J., & Rahmann, S. (2012). Bioinformatics, 28 (19)) for automation and to ensure reproducibility.

Mapping and quantification of the raw reads are performed on a transcript level. Gene indexing is done by the Salmon package. Cell debarcoding, deduplication, read mapping, and estimation of transcript-level expression by pseudo-alignment is done using the Salmon alevin software.

For Quality control (QC) of the raw reads, the software MultiQC (Ewels, P., Magnusson, M., Lundin, S., & Käller, M. (2016). Bioinformatics, 32(19)) is used. QC of the quantification step is done by the package AlevinQC (Charlotte Soneson and Avi Srivastava (2021). https://github.com/csoneson/alevinQC). The Seurat (Satija, R., Farrell, J. A., Gennert, D., Schier, A. F., & Regev, A. (2015) and Scater (McCarthy, D. J., Campbell, K. R., Lun, A. T. L., & Wills, Q. F. (2017). Bioinformatics, 33(8)) packages are used to perform quality control and visualization of the data on the sample-, cell- and gene-level. Included is the detection and removal of outlier cells based on transcript and gene metrics, detection of possible doublet cells and batch effects.

Dimension reduction is done by selecting highly variable genes that account for the most variation in a cell population. The selected features are then used to train ScaiNet for sample classification.

Patient samples were divided into two groups, consisting of those patients that were diagnosed during surgery with endometriosis and with non-endometriosis. Non-endometriosis is defined as patients in which no endometriotic lesions were found during laparoscopy or were disconfirmed by pathology. This resulted in 16 endometriosis samples and 16 non-endometriosis samples.

Approximately 40% of samples from each group were set aside to use for model validation. The remaining 60% of samples were used to train a series of ScaiNet neural networks to distinguish between the endometriosis and non-endometriosis groups. 50 to 100 such networks were trained with a range of chosen hyperparameters.

A gene signature was discovered through pseudobulk analysis of all genes in each cluster of the dataset of endometriosis versus non-endometriosis samples. The analysis showed that the JUP gene was differentially expressed between the CD16+ monocytes of endometriosis versus non-endometriosis samples with a log fold change of 1.21 and a p value of 3.3e10⁻⁵ (FIG. 1).

### Example 2

Specific subsets of monocytes were identified to be expressing the JUP biomarker gene predictive of endometriosis. Cell cluster analysis showed the localization of the differential expression to be confined with CD16+ monocytes which represent 3.6% of the total PBMCs and account for 3927 cells.

### Example 3

Sera obtained from an independent cohort of 67 women with confirmed endometriosis (n=35) or without endometriosis (n=32) were analysed through ELISA (Anawa, MBS2018947-96) for the detection of secreted and soluble JUP. JUP was found to be significantly increased in endometriosis patients (FIG. 2A; p=0.044, Mann Whitney test). The ROC curve presented an AUC of 0.64 which was significantly different from 1 (p=0.006, t-test) (FIG. 2B), making JUP a diagnostic marker for endometriosis.

A lower (>180 ng/ml), an optimal (>259 ng/ml) and a higher (>380 ng/ml) threshold for JUP were identified to be indicative of the disease presence with different accuracies (FIG. 2C, D, E). The low JUP threshold (>180 ng/ml) was defined by the minimal acceptable specificity of 50% (95% CI of 31.2-65.6%). Using such a threshold, the test presented a sensitivity of 65.7% (95% CI of 52.8-84.4%). In the analyzed cohort (endometriosis n=35 and non-endometriosis patients n=32), the low JUP threshold detected 22 of the endometriosis patients and 15 of the non-endometriosis patients (FIG. 2C).

The optimal JUP threshold (>259 ng/mL) was defined by the method of the “closest top left corner” of the ROC curve. Using such a threshold, our assay presented a sensitivity of 51.4 (95% CI of 37.1-65.7%) and specificity of 81.2% (95% CI of 65.6-93.8%). In the analyzed cohort (endometriosis n=35 and non-endometriosis patients n=32), the optimal JUP threshold detected 18 endometriosis patients and 6 non-endometriosis patients (FIG. 2D).

The maximal JUP threshold (>380 ng/ml) was defined by the highest specificity. Using such a threshold, our assay presented a sensitivity of 31.4% (95% CI of 17.1-48.6%) and specificity of 100% (95% CI of 100-100%). In the analyzed cohort (endometriosis n=35 and non-endometriosis patients n=32), the maximal JUP threshold detected 11 endometriosis patients and 0 non-endometriosis patients (FIG. 2E).

The levels of JUP were modestly affected by the menstrual cycle phase but a significant difference between endometriosis and non-endometriosis patients was found during the secretory phase (p=0.005, Mann Whitney test). Indeed, using the lowest acceptable JUP threshold (>112 ng/ml), a sensitivity of 100% (95% CI of 100-100%) and a Specificity of 55.6% (95% CI of 22.2-83.6%) were achieved in patient samples collected during the secretory phase (FIG. 2F). Using an optimal JUP threshold defined in the secretory phase (>256 ng/ml), a sensitivity of 77.8% (95% CI of 49.7-100%) and specificity 85.7% (95% CI of 57.1-100) were achieved in the same cohort (FIG. 2G). Using the maximal JUP threshold (>380 ng/ml), a higher sensitivity of 55.6% (95% CI of 22.2-83.6%) and a Specificity of 100% (95% CI of 100-100%) were achieved in patient samples collected during the secretory phase (FIG. 2H).

The inventors detected the following characteristics in the studied population of patients with endometriosis and high levels of JUP (>380 ng/ml): No difference in pain (dysmenorrhea) or age; lower proportions of endometrioma, peritoneal endometriosis or low stage endometriosis; higher percentage of severe endometriosis or DIE (versus patients with endometriosis and low levels of JUP) (FIG. 2I).

The inventors detected high levels of JUP (>380 ng/ml) in late or severe endometriosis defined by rASRM stage of the disease or ENZIAN score when available, suggesting a role of JUP as prognostic endometriosis marker (FIG. 2J). Indeed, different thresholds of detectable JUP in the serum of patients might be used as a reference pattern to the progression of the disease. With a minimum sensitivity of 21.4% (71.0-39.5%) using the highest JUP cutoff and a maximum of 64.3% (42.9-85.7%) using the lowest JUP cutoff, JUP was performing as well in early stages of the disease.

In conclusion, soluble JUP is a novel biomarker for diagnosis and prognosis of endometriosis.

### Example 4

CA125 (DRG, EIA-5072), HE4 (Fujirebio AB; 404-10), IL-6 (Biotechne, HS600C), TNF-alpha (Biotechne HSTA00E) and CRP (Biotechne BAM17072; Sigma, C3527) were tested by ELISA to determine their contribution to the diagnostic and prognostic power of JUP in endometriosis via protein quantification of patients' sera.

CA125 was found to be significantly increased in endometriosis patients (FIG. 3A; p=0.0003, Mann Whitney test) and especially in high grade endometriosis (p=0.0009, Mann Whitney test). The ROC curve had an AUC of 0.74 which was significantly different from 1 (p=0.014, t-test) (FIG. 3B). The inventors identified a lower (13.8 U/ml), an optimal (16.8 U/ml), and a higher (35 U/ml) CA125 threshold.

Using the higher cutoff of 35 U/ml, CA125 identified 11 women out of 67 of the analysed cohort. Among them, 9 had confirmed endometriosis while 2 were false positives, leading to a specificity of the biomarker of 93.8% (95% CI of 84.4-100%) and a sensitivity of 25.7% (95% CI of 12.9-40.1%) (FIG. 3A).

At their respective optimal cutoffs, JUP had better specificity but lower sensitivity than CA125 (for JUP>259 ng/ml, sensitivity 51.4% and specificity 81.2%; for CA125>16.8 U/ml, sensitivity 77.1% and specificity 75.0%).

At their respective highest cutoffs, JUP had better specificity but lower sensitivity than CA125 (for JUP>380 ng/ml, sensitivity 31.4% and specificity 100%; for CA125>35 U/ml, sensitivity 25.7% and specificity 93.8%).

A combination of JUP with CA125 with each at the highest cutoff identified 14 out of 35 endometriosis patients and 2 additional false positive patients, and thereby could increase the sensitivity to 40% and the specificity to 93.8% (FIG. 3C).

The complementarity of both markers is apparent by the ability of each marker to detect different endometriosis subtypes. The population of patients with high CA125 (>35 U/mL) but low JUP (<380 ng/mL) includes cases with Endometrioma (40%, 2 out of 5 patients), cases with high grade endometriosis (IV, 40%, 2 out of 5 patients) and no cases with peritoneal endometriosis. The population of patients with low CA125 (<35 U/mL) but high JUP (>380 ng/mL) includes a higher proportion of Peritoneal Endometriosis (40%, 2 out of 5 patients) and low grade endometriosis (rASRM I, 20%, 1 out of 5 patients; II, 20%, 1 out of 5 patients; III, 20%, 1 out of 5 patients). This population did not have any cases of endometrioma (Table 2).

Thus, while JUP alone is particularly useful as a diagnostic marker of endometriosis in early and peritoneal endometriosis, the addition of CA125 detection improves the sensitivity of our assay by including more patients with severe forms of endometriosis (DIE and rASRM IV) and extending its utility to other subtypes (endometrioma).

HE4 was measured in the sera of endometriosis and non-endometriosis (CTL) patients to determine JUP as a specific marker for diagnosis and prognosis of endometriosis and not ovarian cancer. HE4 measurements were not affected by the endometriosis condition of our patient cohort in line with the current literature claiming HE4 to be a specific marker for ovarian cancer (FIG. 4A).

TNF-alpha, IL-6 and CRP were measured in the sera of endometriosis and non-endometriosis (CTL) patients to determine the specificity of JUP as a marker for diagnosis and prognosis of endometriosis. Data analysis showed that JUP and the studied inflammatory markers did not consistently associate. Hence, high JUP levels could be identified in subgroups of endometriosis patients with low inflammatory marker levels. All in all, the inventors found that the JUP level did not associate with a specific inflammatory signature (FIG. 4B, C, D).

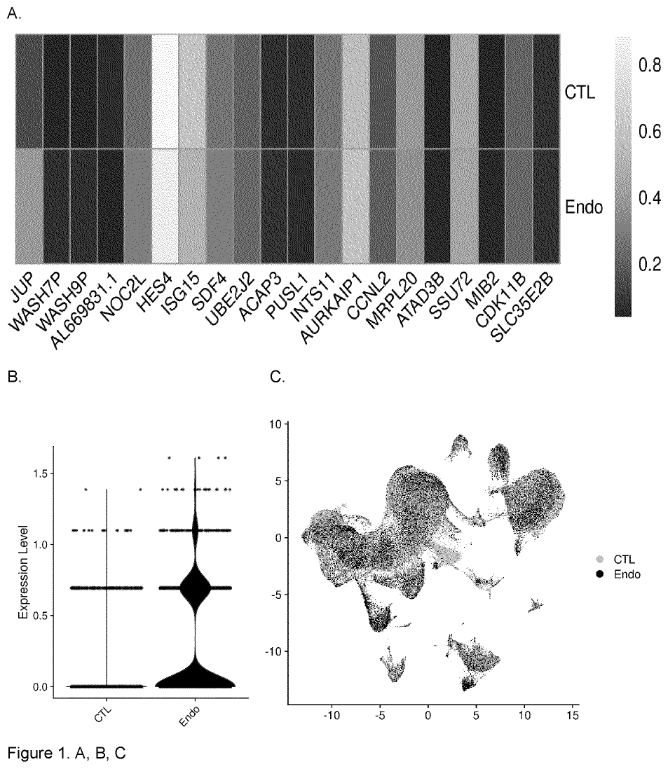

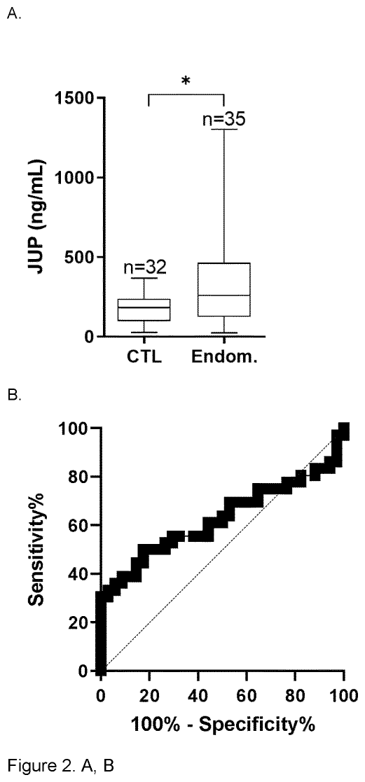

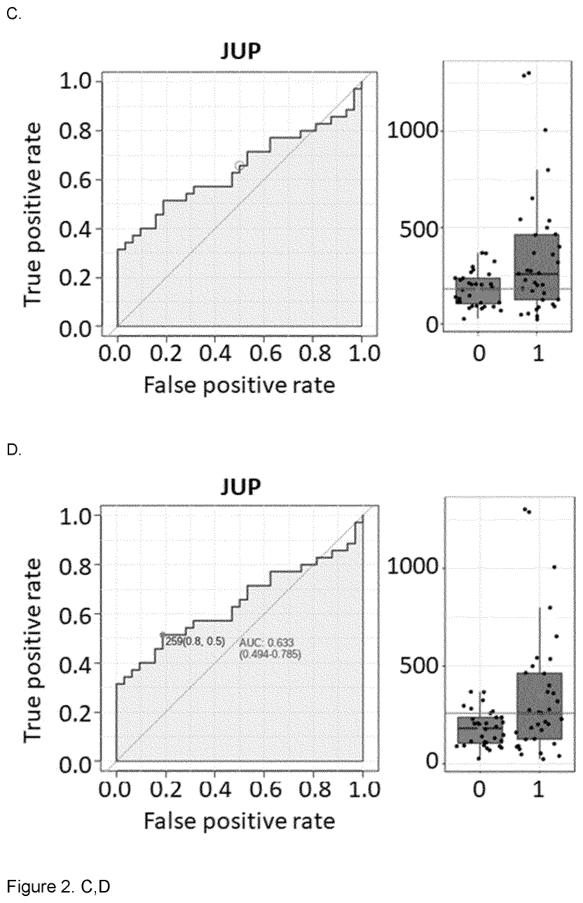

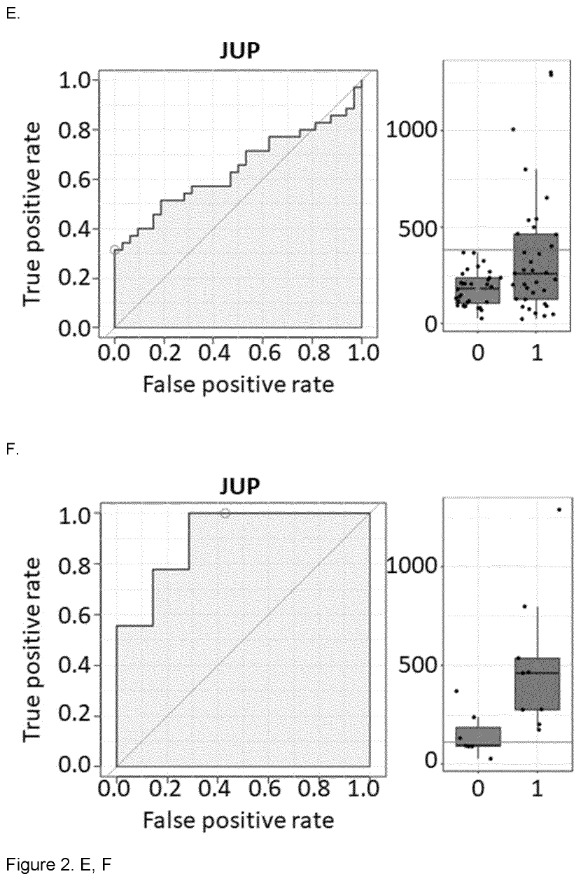

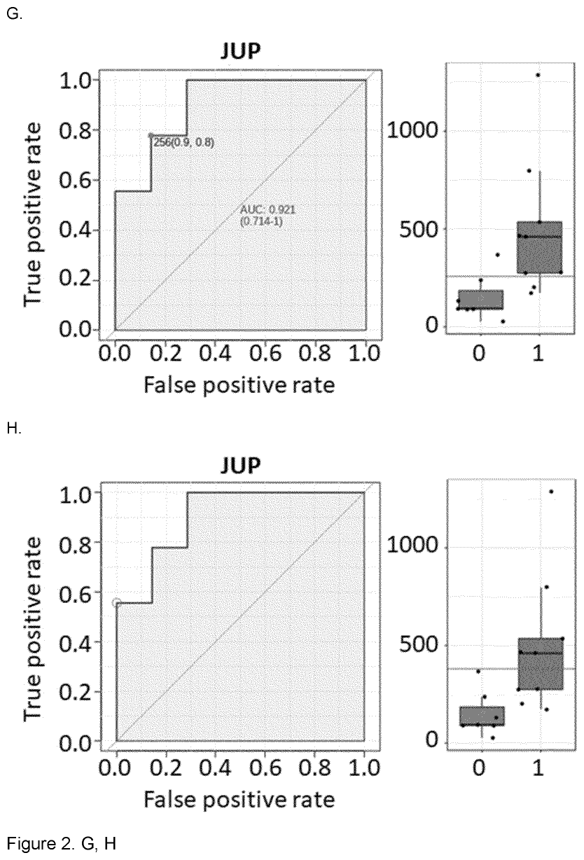

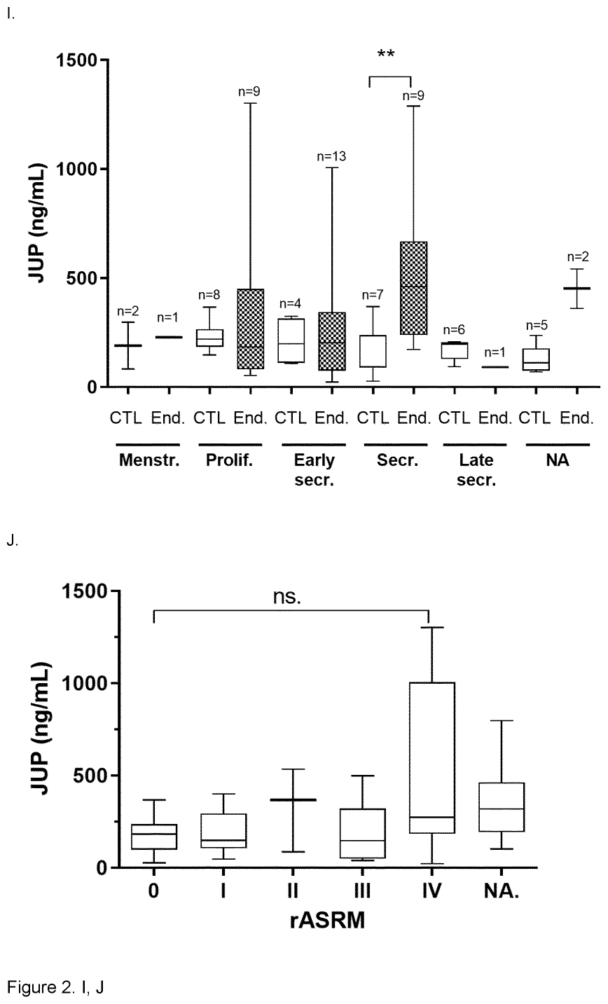

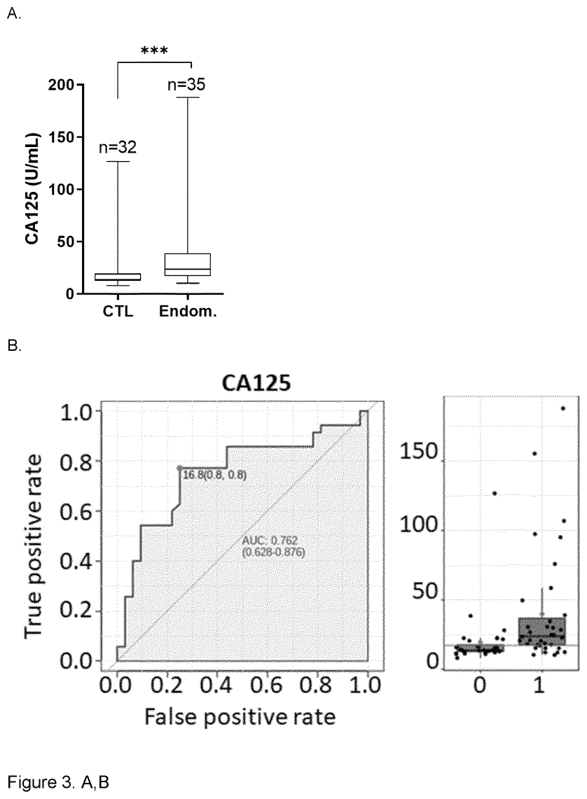

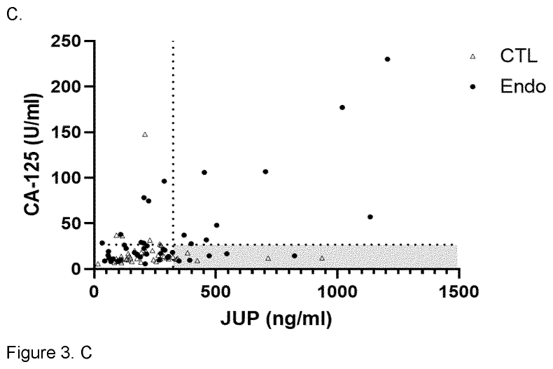

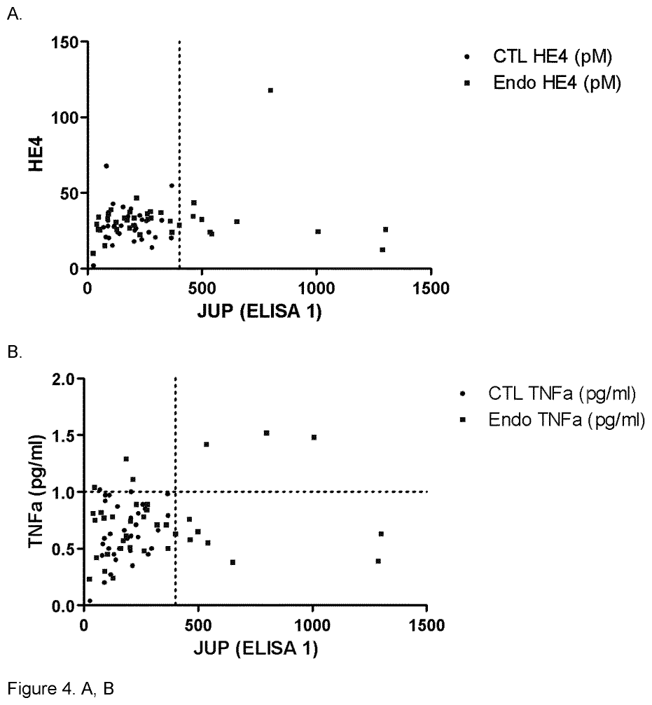

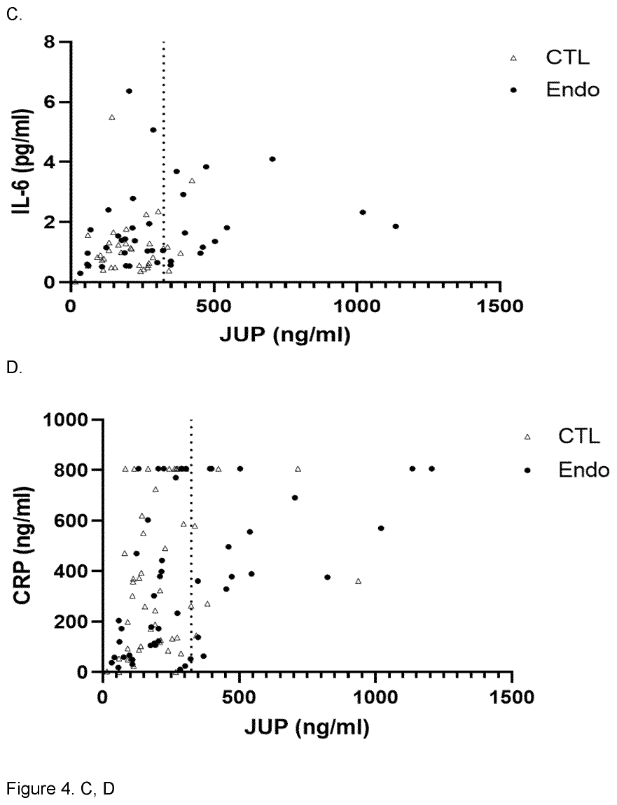

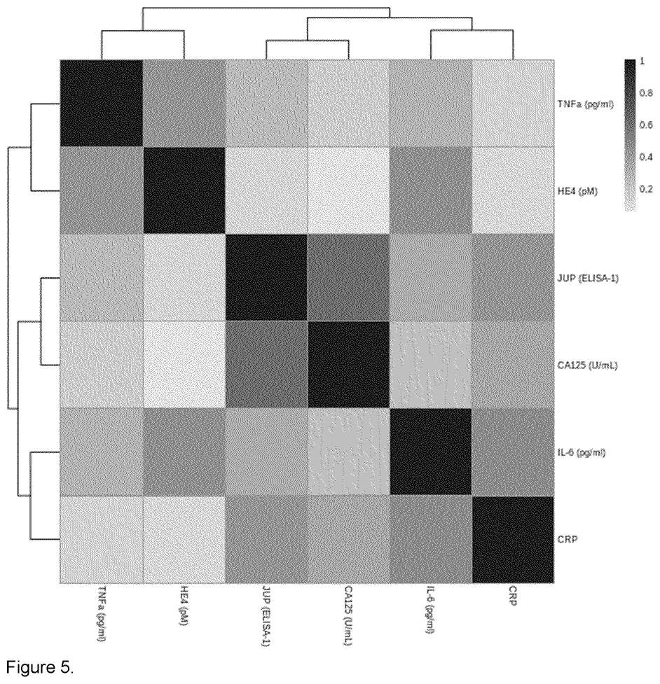

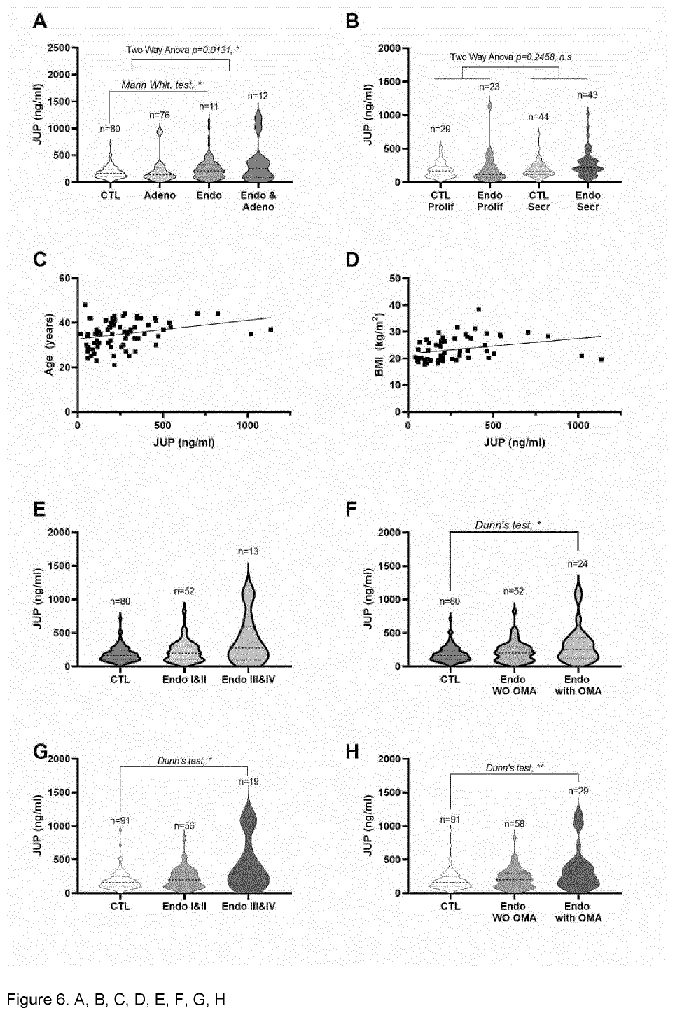

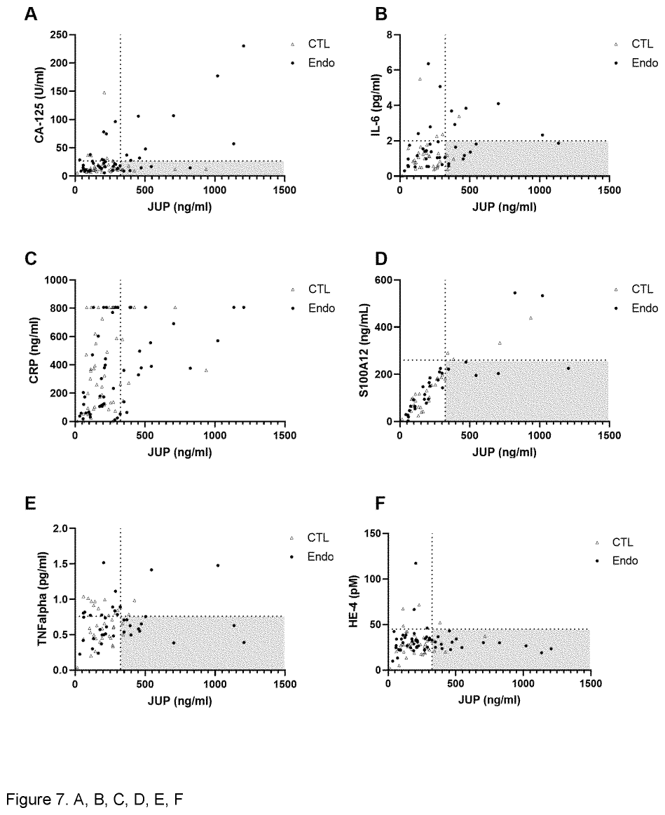

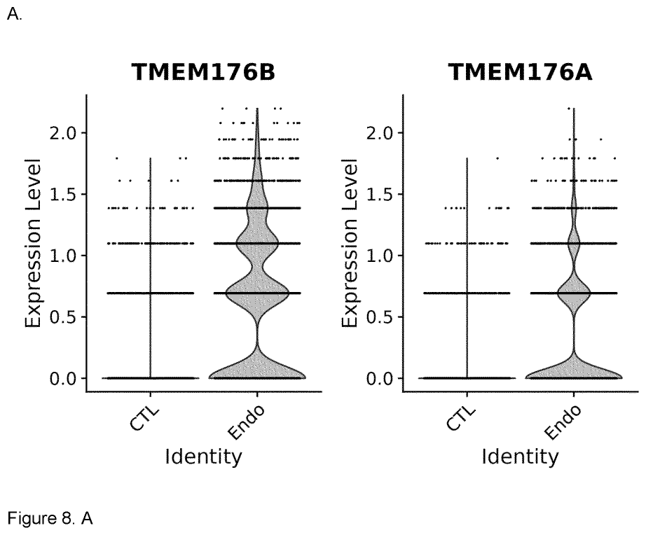

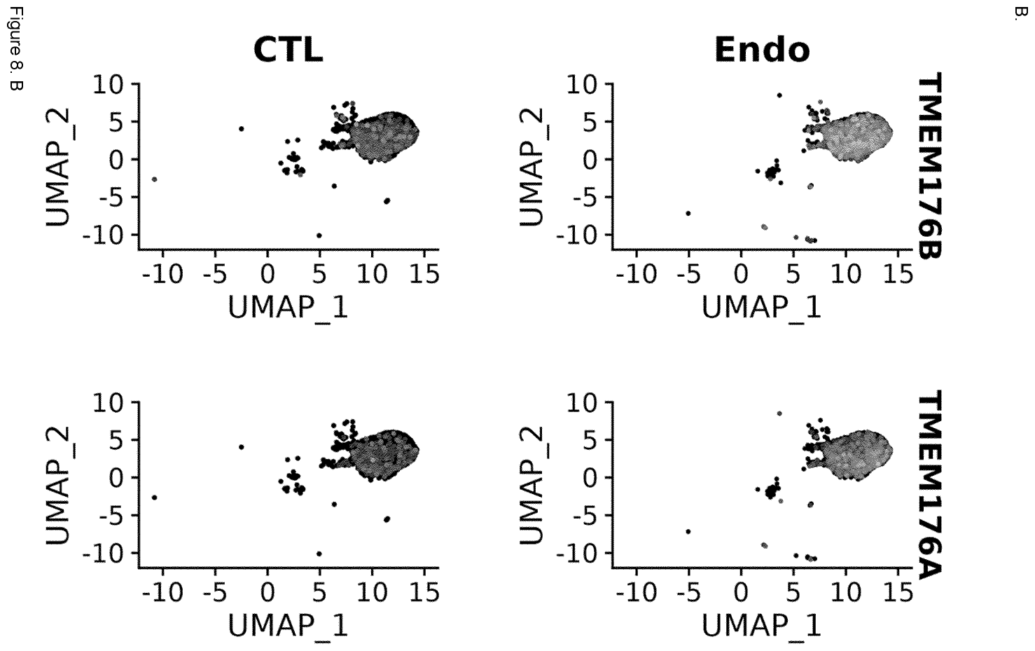

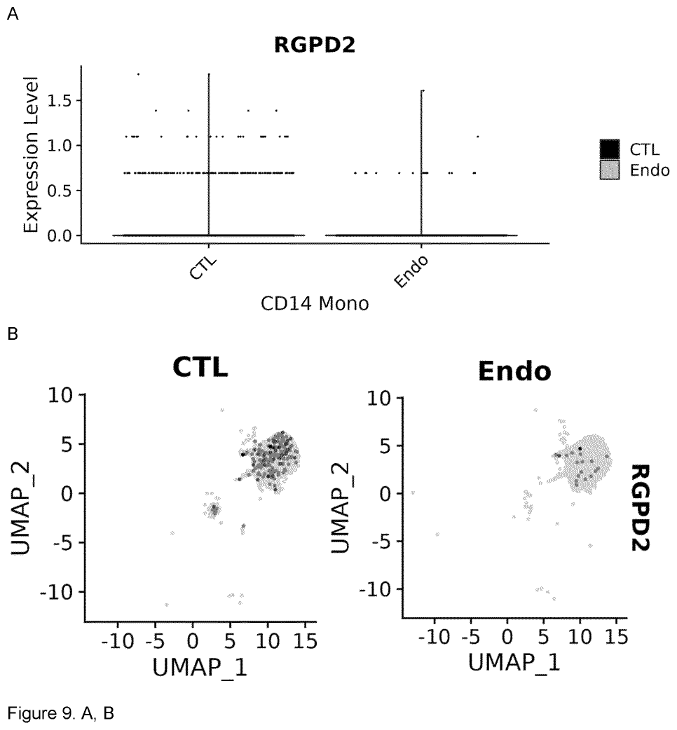

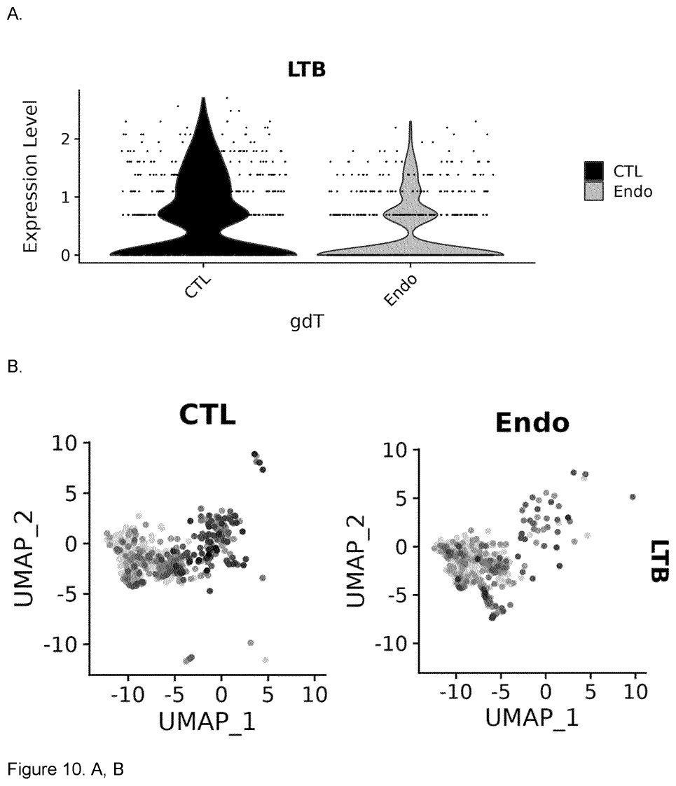

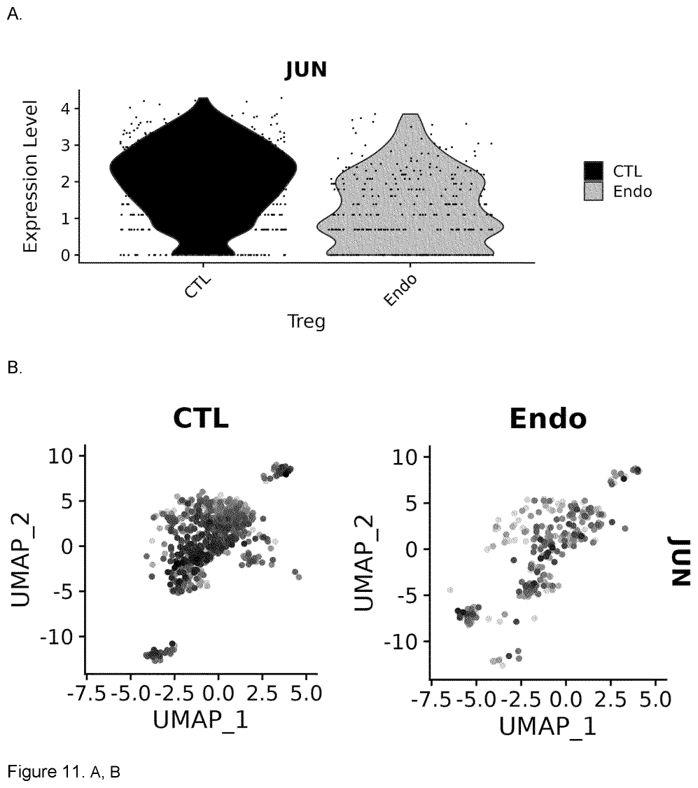

JUP tended to positively correlate with CA125, IL-6 and CRP and to negatively correlate with HE4 (FIG. 5).

TABLE 1
Soluble biomarkers predicting Endometriosis

CA125
HE4
IL-6
TNF-a
CRP
S100A12

TABLE 2
Correlation of various covariates on serum with JUP.

| | CTL | | | Endo | | |
|---|---|---|---|---|---|---|
| | Pearson's | P | Total number | Pearson's | P | Total number |
| | r | value | of values | r | value | of values |
| Age | −0.062 | 0.587 | 80 | 0.273 | 0.017 | 76 |
| BMI | 0.221 | 0.084 | 62 | 0.292 | 0.027 | 57 |
| Cycle day | 0.103 | 0.380 | 75 | 0.010 | 0.937 | 66 |
| Dysmenorrhea | 0.083 | 0.509 | 66 | 0.127 | 0.286 | 72 |
| Abdominal- | 0.070 | 0.571 | 68 | 0.151 | 0.198 | 74 |
| pain | | | | | | |
| Dyschezia | −0.052 | 0.717 | 51 | −0.010 | 0.938 | 61 |
| Dysuria | −0.015 | 0.919 | 51 | 0.039 | 0.765 | 61 |
| Dyspareunia | −0.159 | 0.210 | 64 | −0.017 | 0.886 | 71 |
| Progesterone | −0.076 | 0.511 | 76 | 0.093 | 0.434 | 73 |
| levels- | | | | | | |
| Serum | | | | | | |
| Progesterone | 0.178 | 0.248 | 44 | −0.036 | 0.813 | 46 |
| levels- | | | | | | |
| Peritoneal | | | | | | |
| Fluid | | | | | | |
| Relative OP | −0.004 | 0.979 | 49 | −0.022 | 0.877 | 50 |
| time | | | | | | |

### Example 5

### Planned Validation

An independent validation cohort of patients will be recruited, consisting of a similar patient cohort as recruited in the study before. The endometriosis status of all samples in the validation cohort will be determined using an optimized multiplexed serum protein measurement of part or all the markers of Table 1. Characterization of each biomarker and their performances will be thoroughly assessed in several human tissue types to provide a comprehensive, specific and sensitive assay for the diagnosis and/or prognosis of endometriosis, and to infer or exclude the diagnosis of other diseases or disorders of the female reproductive tract (e.g. ovarian and endometrial cancer). A full reference panel of JUP and the relevant markers will be built across stages of endometriosis to identify optimal thresholds of the biomarkers in Table 1 for the prognosis of the disease. Using serum protein measurements in combination with data from the clinical characteristics of each patient, we will achieve higher accuracies and performances. Using nucleotide measurements (e.g. RNA), we will extend and specialize the use of our panel. Comparisons with other methods used in diseases or disorders of the female reproductive tract will be performed. Finally, our invention will be benchmarked against the gold standard method for diagnosis and treatment of diseases or disorders of the female reproductive tract (i.e. surgical laparoscopy for endometriosis).

### Example 6

The inventors further analyzed an expanded cohort of samples. Sera samples were obtained from 178 patients undergoing gynecologic laparoscopy for benign indications between November 2013 and September 2021. Indications for surgery included pelvic pain, infertility, chromopertubation, ovarian cysts, sterilization, salpingectomy hysterectomy and endometriosis diagnosis. JUP serum level was evaluated in function of endometriosis, severity and type of, adenomyosis, menstrual cycle and various other covariates. The patients were allocated in the proliferative phase or in the secretory phase according to self-reported menstruation dates and measured progesterone levels.

Serum JUP concentrations were significantly elevated in women with endometriosis (Two way Anova, p=0.0131) (FIG. 6A) but not the presence of adenomyosisand globally, not affected by the menstrual phase (Two way Anova, p=0.2458). In contrast to other endometriosis markers such as CA125 (Kafali, H., Artuc, H., & Demir, N., 2004, European Journal of Obstetrics & Gynecology and Reproductive Biology, 116(1), 85-88) or IL6 (Matthias W. Angstwurm et al. Cytokine. 1997 May; 9(5):370-4.), serum JUP was not significantly affected neither by cycle day nor by progesterone level (Table 2).

Serum JUP was positively correlated with age (Pearson's r=0.273; p=0.017, *, n=76) and BMI (Pearson's r=0.292; p=0.027, *, n=57) in the endometriosis group (FIG. 6C-D, Table 2). This association, rather weak (Pearson's r<0.3), should be considered in the perspective of the severity of endometriosis, with the understanding that older patients have a higher BMI and are more likely to undergo surgery for OMA removal and severe endometrosis. In fact, patients with severe endometriosis tend to have higher JUP levels (FIG. 6E, One Way Anova, p=0.103, control group n=80, rASRM I&II n=52 and rASRM III&IV n=13) and JUP levels were significantly increased in presence of endometrioma (OMA) (FIG. 6F, One Way Anova, p=0.0210, control group n=81, endometriosis without OMA n=51 and endometriosis with OMA n=24). Since adenomyosis was not identified as a risk factor for serum JUP expression (FIG. 6A), adenomyosis patients were included to the pool to increase the power of the analysis (total patient number in the control group=91, in the endometriosis group with known rASRM score=75 and endometriosis group with characterized lesions=86). In this configuration, serum JUP level was significantly increased in severe endometriosis (One Way Anova, p=0.0184) (FIG. 6G) and again in presence of OMA (One Way Anova, p=0.008) (FIG. 6H).

These extended results confirm a link between serum JUP and endometriosis, especially with severe endometriosis and with endometrioma. In contrast, it was not possible to find a statistical link between serum JUP and cycle phase, pain and/or adenomyosis.

### Example 7

The inventors further analyzed the expanded cohort of samples and compared the biomarker JUP to the biomarkers used previously in endometriosis. The inventors correlated JUP with the inflammatory markers CRP, and IL-6 and with the endometriosis markers CA125 and S100A12. The strength of the linear association between JUP and CRP or IL-6 was relatively weak (Pearson's r<0.4) indicating that different populations are identified. The association was stronger with CA125 and S100A12 (Pearson's r 0.5 and 0.8 respectively). CA125 and JUP were mostly upregulated in endometriosis patients but interestingly both molecules recognized different populations (FIG. 7A; The gray area highlights the patient recognized by JUP but not by CA125). The same applies to IL-6 and S100A12, TNFalpha and HE-4 wherein, depending on the threshold, certain individuals can only be identified by JUP (FIG. 7B, 7D, 7E, 7F). These results suggested a complementarity of CA125 and JUP in endometriosis identification

### Example 8

Serum JUP used as a stratification marker for identifying endometriosis related genes in PBMCs.

To discover a gene signature for endometriosis generated scRNA-seq data from endometriosis free women (n=10) and women with endometriosis (n=10) were compared. The analysis highlighted ribosomal protein S26 in Hematopoietic stem and progenitor cells (HSPC) as potential differentially expressed genes in endometriosis with a log fold change of 3.61 (p value 0.06). A decrease of lymphotoxin beta, a cytokine of the type II membrane protein of the TNF family, was also noticed in Gamma delta T cells (gdT, log fold change of −0.77, p value 0.54).

Given the large number of deregulated genes associated with serum JUP, we considered evaluating its ability as a stratification marker. The comparison of the scRNA-seq data generated from endometriosis free women (n=5) and women with endometriosis (n=6) with low serum JUP values identified 3 DGs (TRBV2 in CD4 CTL; TMEM176A and TMEM176B in CD14+ Monocytes) (“DGs” stands for “Dysregulated Genes”). Similar approach in women with low serum JUP values (endometriosis free women n=5; women with endometriosis n=4) identified 9 DGs in high JUP group (HBB, HBA1, HBA2, RGPD2 in CD14 Monocytes; CH25H, HBB, LTB, KLRC1 in gdT; IFIT2 in NK CD56bright and JUN in Treg). Aside from KLRC1 which was up-regulated, all other DGs were down-regulated. (see FIGS. 8-11)

1. A method for diagnosing a disease or disorder of the female reproductive tract, the method comprising the steps of:
i) determining a protein level of the biomarker JUP in a sample of a female subject; and
ii) diagnosing a disease or disorder of the female reproductive tract based on the determination of i), preferably wherein a protein level above 112 ng/ml serum-equivalent of the biomarker JUP is indicative for a disease or disorder of the female reproductive tract.

2. A method for predicting disease development, disease progression and/or disease outcome of a female subject having a disease or disorder of the female reproductive tract or at risk of having a disease or disorder of the female reproductive tract, the method comprising the steps of:
i) determining a protein level of the biomarker JUP in a sample of a female subject;
ii) comparing the level determined in i) to a prediction reference pattern; and
iii) predicting disease development, disease progression and/or disease outcome of the female subject based on the comparison in step ii), preferably wherein a protein level of the biomarker JUP above the prediction reference pattern is indicative for more likely disease development, more likely disease progression and/or worsening of disease outcome.

3. A method for predicting susceptibility to a treatment for a disease or disorder of the female reproductive tract of a female subject having a disease or disorder of the female reproductive tract or at risk of having a disease or disorder of the female reproductive tract, the method comprising the steps of:
i) determining a protein level of the biomarker JUP in a sample of a female subject;
ii) comparing the level determined in i) to a susceptibility reference pattern; and
iii) predicting susceptibility to a treatment for a disease or disorder of the female reproductive tract of the female subject based on the comparison in step ii), preferably wherein a level of the biomarker JUP above the susceptibility reference pattern is indicative for increased susceptibility to a treatment for a disease or disorder of the female reproductive tract.

4. The method of claim 3, wherein the treatment for a disease or disorder of the female reproductive tract is an endometriosis treatment selected from the group of: pain medication, hormonal therapy, fertility treatment and surgery.

5. The method of claim 1 to 4, wherein a level of at least the biomarker CA125 is additionally determined in i), preferably wherein the level of the biomarker CA125 is a protein level.

6. The method of claim 1 to 5, wherein a level of at least 2, 3, 4, or 5 biomarkers is determined.

7. The method of claim 6, wherein determining the level of biomarkers comprises determining at least one biomarker using a nucleic acid detection technique.

8. The method of claim 6 or 7 wherein at least one of the biomarkers is selected from the group consisting of: HBA1, HBA2, TRBV2, HBB, TMEM176A, TMEM176B, LTB, KLRC1, RGPD2, IFIT2, JUN and CH25H, preferably wherein the biomarkers comprise at least one biomarker selected from the group consisting of:
a) TRBV2 in CD4+ cytotoxic T cells;
b) TMEM176A and TMEM176B in CD14+ monocytes;
c) HBB, HBA1, HBA2, RGPD2 in CD14+ monocytes;
d) CH25H, HBB, LTB, KLRC1 in gamma delta T cells;
e) IFIT2 in CD56+ natural killer cells; and
f) JUN in regulatory T cells.

9. The method of any one of claims 1 to 8, wherein sample of a female subject is a sample selected from the group of endometrium sample, menstrual blood sample, vaginal smear sample, a blood sample and/or a cervical smear sample.

10. The method of any one of claims 1 to 9, wherein the method is at least partially computer-implemented and wherein the level(s) of the biomarker(s) is/are determined by retrieving data indicative for the level(s) of the biomarker(s).

11. The method of any one of claims 1 to 10, wherein the method additionally comprises determining at least one non-molecular marker, preferably wherein the non-molecular marker comprises a marker selected from the group consisting of: age, weight, BMI, gravidity, parity, ethnicity, dysmenorrhea, fertility status, previous laparoscopies, previous use of medication and other gynaecological disorders.

12. The method of claim 11, wherein at least 2 biomarkers are determined and wherein the biomarkers comprise or consist of the biomarkers CA125 and JUP, preferably wherein at least 3 biomarkers are determined, wherein the biomarkers comprise or consist of the molecular biomarkers CA125 and JUP and the non-molecular marker dysmenorrhea.

13. The method for predicting of any one of claims 2 to 12, wherein the reference pattern is obtained from reference subjects, wherein at least one of the reference subjects is diagnosed with a disease or disorder of the female reproductive tract.

14. The method for predicting of claim 13, wherein obtaining the reference pattern from reference subjects comprises a machine-learning technique, preferably a convolutional neural network and/or logistic regression.

15. A method for classification of a female subject having a disease or disorder of the female reproductive tract or at risk of having a disease or disorder of the female reproductive tract into a class, the method comprising the steps of:
a) i) determining a level of the biomarker JUP in a sample of a female subject;
ii) predicting disease development, disease progression and/or disease outcome of a female subject according to the method of claim 2, 5 to 14; and/or
iii) predicting susceptibility to a treatment for a disease or disorder of the female reproductive tract of a female subject according to the method of any one of claims 3 to 14; and
b) classifying the female subject according to the level determined in i), prediction of ii), and/or prediction in iii).

16. The method of claim 15, wherein at least one class is indicative of the stage, type and/or severity of the endometriosis.

17. Use of a composition comprising reagents for the detection of the biomarker JUP for the diagnosis of a disease or disorder of the female reproductive tract,

18. Use of the composition of claim 17, wherein the disease or disorder of the female reproductive tract is endometriosis.

19. Use of the composition of claim 17 or 18, wherein the composition further comprises reagents for the detection of CA125.

20. A pharmaceutical product comprising a compound against a disease or disorder of the female reproductive tract for use in treatment of a female subject that is predicted as susceptible to a treatment for a disease or disorder of the female reproductive tract according to the method for predicting susceptibility to a treatment of claim 3 to 16.

21. The pharmaceutical product of claim 20, wherein the compound against a disease or disorder of the female reproductive tract is selected from the group of: ibuprofen, naproxen, oxycodone, desogestrel, dienogest, levonorgestrel, clomiphene citrate, gonadotropins, metformin, letrozole and bromocriptine.

22. The method of any one of the claims 1 to 16, the use of the composition of any one of the claims 17 to 19 or the pharmaceutical product of claim 20 or 21, wherein the disease or disorder of the female reproductive tract is endometriosis or ovarian cancer.

23. The method of claim 22, the composition of claim 22 or the pharmaceutical product of claim 22, wherein the disease or disorder of the female reproductive tract is endometriosis.

24. The method of claim 23, the composition of claim 23 or the pharmaceutical product of claim 23, wherein the endometriosis is selected from the group of: peritoneal endometriosis (SUP), endometrioma(s), deeply infiltrating endometriosis (DIE) and abdominal wall endometriosis.

25. The method of claim 23 or 24, the composition of claim 23 or 24 or the pharmaceutical product of claim 23 or 24, wherein the endometriosis is at rASRM II, III, or IV, preferably wherein the endometriosis is at rASRM IV stage.

26. A computer program product comprising instructions to execute the method of any one of claims 13 to 16 or 22 to 25, wherein the method is a computer-implemented method and wherein the level of the biomarker JUP in a sample of a female subject is retrieved instead of determined.

27. A kit comprising reagents for the detection of JUP protein and for the detection of at least one RNA marker selected from the group consisting of: HBA1, HBA2, TRBV, HBB, TMEM176A, TMEM176B LTB, KLRC1, RGPD2, IFIT1, JUN and CH25H, preferably wherein the RNA biomarker(s) comprise or consist of at least one biomarker selected from the group consisting of:
a) TRBV2 and CD4;
b) TMEM176A and CD14;
c) i) HBB, HBA1, HBA2 and/or RGPD2; and ii) CD14+;
d) i) CH25H, HBB, LTB and/or KLRC1 and ii) a gamma delta marker;
e) IFIT2 and CD56; and
f) JUN and a regulatory T cell marker.

28. The kit of claim 27 further comprising an instruction manual how to employ the method of any one of the claims 7-16, or 22-25.
